(19) United States
(12) Patent Application Publication
(10) Pub. No.: US 2012/0225072 A1
(43) Pub. Date: Sep. 6, 2012

(54) STABLE FORMULATIONS OF IMMUNOGLOBULIN SINGLE VARIABLE DOMAINS AND USES THEREOF

(75) Inventors: Yves Meyvis, Gent (BE); Ann Brige, Ertvelde (BE)

(73) Assignee: Ablynx N.V., Zwijnaarde (BE)

(21) Appl. No.: 13/409,661

(22) Filed: Mar. 1, 2012

Related U.S. Application Data
(60) Provisional application No. 61/448,586, filed on Mar. 2, 2011.

(51) Int. Cl. A61K 39/395; C07K 1/00; C07K 16/46; C07K 16/28; G01N 33/566

(52) U.S. Cl. 424/136.1; 530/387.3; 435/7.92; 436/501; 424/133.1

(57) ABSTRACT

The present invention relates to stable formulations of polypeptides, e.g. immunoglobulin single variable domains.

### RELATED APPLICATIONS

This application claims the benefit under 35 U.S.C. §119(e) of U.S. provisional patent application Ser. No. 61/448,586, filed Mar. 2, 2011, the contents of which are incorporated herein by reference in its their entirety.

### FIELD OF THE INVENTION

The present invention relates to stable formulations of polypeptides, e.g. immunoglobulin single variable domains.

### BACKGROUND OF THE INVENTION

Immunoglobulin single variable domains, such as camelid VHH domains, camelized VH domains or humanized VHH domains, represent a rapidly growing class of therapeutics. For example, immunoglobulin single variable domains against CXCR4 have been described in WO 09/138,519, U.S. Ser. No. 61/358,495, PCT/EP2011/050156 and PCT/EP2011/050157.

Immunoglobulin single variable domains typically must be stored and transported between initial manufacture and use, e.g. administration to a patient. Transport, manufacture and storage can exert manifold stresses on the immunoglobulin single variable domain, such as chemical and physical stresses. Chemical stresses may cause deamidation, racemization, hydrolysis, oxidation, beta-elimination, pyroglutamate formation or disulfide exchange. Physical stresses can cause denaturation, aggregation, precipitation or adsorption.

It is known that these stresses can affect physicochemical integrity of protein therapeutics, e.g. antibody therapeutics. For example, aggregation, deamidation and oxidation have been described as most common causes of antibody degradation (Cleland et al., 1993, Crit. Rev. Ther. Drug Carrier Systems 10, 307-377). At the same time it is instrumental that formulations are provided which preserve chemical and physical integrity of the immunoglobulin single variable domains. Chemical and physical integrity are required for use e.g. as a therapeutic agent, and typically are also associated with biological activity.

Little is known about suitable formulations of immunoglobulin single variable domains. WO 10/077,422 describes a formulation of a TNF binding Nanobody comprising a lyoprotectant, surfactant and a buffer chosen from histidine buffer and Tris buffer at a pH between 5.0 to 7.5. The formulation is described as advantageous for a specific TNF-binding Nanobody construct. The unpublished copending application of the present applicant PCT/EP2010/062975 describes a formulation for e.g. specific IL6R specific immunoglobulin single variable domain based therapeutics comprising an aqueous carrier having a pH of 5.5 to 8.0, a buffer such as histidine, HEPES, MES, succinate or acetate, an excipient and a surfactant. The unpublished copending application of the present applicant PCT/EP2010/062972 describes a formulation of a specific immunoglobulin single variable domain polypeptide sequence, which has a pH between 5.5 and 7.0, and comprises a phosphate or acetate buffer. Each of these documents describes particular advantages for different formulations in the context of specific embodiments of immunoglobulin single variable domain based therapeutics.

### SUMMARY OF THE INVENTION

There remains a need for providing formulations for immunoglobulin single variable domains, e.g. as defined herein, which enhance thermal stability, preserve the active agent against chemical and mechanical stress, and hence allow storage and temperature changes without physical or chemical deterioration, and remain stable for prolonged periods of time.

The present invention relates to stable formulations of polypeptides, e.g. immunoglobulin single variable domains, in particular immunoglobulin single variable domains directed against CXCR4, such as immunoglobulin single variable domains according to Seq Id No.: 2 to 5, specifically Seq Id No.: 2, i.e. the Nanobody 4CXCR104.

The invention provides formulations which are stable upon storage for prolonged periods of time and over a broad range of temperatures e.g. 1 to 36 months or more at temperatures between −70° C. and 40° C. The formulations of the invention provide for a high thermal stability of the polypeptide, allow multiple freeze-thaw cycles without chemical or physical deterioration, and provide stability in relation to mechanical stress, such as shake, shear or stir stress. They are suitable for pharmaceutical and diagnostic preparations and compatible with pharmaceutically acceptable diluents, such as saline, certain buffers or water.

The present invention also relates to methods of preparation, methods for storage and uses of the formulations. The invention further relates to dosage unit forms, kits and medical uses of the formulations.

Thus in one aspect, the present invention relates to a formulation comprising a polypeptide binding to CXCR4, characterized in that it comprises a citrate or phosphate buffer and has a pH in the range of 5.0 to 7.5, more specifically a formulation, wherein the polypeptide comprises at least one immunoglobulin single variable domain binding to CXCR4. In particular embodiments the polypeptide comprises at least one nanobody.

In further embodiments of the invention, the polypeptide is half-life extended, e.g. by comprising a polypeptide sequence which binds to serum albumin, which may in some embodiments be an immunoglobulin single variable domain or a fragment thereof capable of binding to serum albumin.

In particular embodiments of the invention the polypeptide binding to CXCR4 comprises at least one of SEQ ID No. 2 to SEQ ID No. 5, preferably SEQ ID No. 2.

In some embodiments of the invention the buffer has a concentration in the range of 5-100 mM, preferably 5-70 mM, more preferably 5-40 mM, e.g. 10 mM, wherein each value is understood to optionally encompass a range of ±5 mM.

In some embodiments the formulation has a pH of 5.0, 5.5, 6.0, 6.5, 7.0 or 7.5, preferably 5.5 to 6.5, more preferably 6.0, wherein each value is understood to optionally encompass a range of ±0.2.

The invention in certain embodiments relates to formulations which are suitable for parenteral administration, such as one or more selected from intravenous injection, subcutaneous injection, intramuscular injection or intraperitoneal injection.

In formulations of the invention, the polypeptide can in certain embodiments have a concentration in the range of 0.1 to 150 mg/ml, preferably 5-50 mg/ml, such as 5, 10, 15, 20, 25, 30, 35, 40, 45 or 50 mg/ml, preferably 10 mg/ml, wherein each value is understood to optionally encompass a range of ±20% of the specific value.

The formulations of the invention may further comprise at least one excipient, which may optionally be one or more selected from the list consisting of NaCl, sucrose or mannitol, e.g. wherein NaCl has a concentration in the range of 10-500 mM, such as 50, 75, 100, 150, 250 or 500 mM, preferably 25-100 mM, e.g. 75-100 mM; and/or mannitol has a concentration of 1-10%, preferably 2-4%, e.g. 2 or 3% (w/w); and/or sucrose has a concentration of 1-12%, preferably 2-7%, e.g. 4, 5 or 6% (w/w).

The invention relates to formulations having an osmolality in the range of 290±60 mOsm/kg.

Particular examples of formulations according to the invention are such wherein the buffer is selected from a) or b)
a) phosphate and preferably has a pH in the range of 6.5 to 7.0, preferably 7.0; or
b) preferably citrate and preferably has a pH between 5.5 and 6.5, more preferably 6.0.

The formulations of the invention may further comprise a non-ionic detergent such as Tween-80, preferably in a concentration between 0.005 and 0.1% w/w, more preferably 0.01%.

In a particular embodiment of the invention, the buffer is a citrate buffer at pH 6.0±0.5, e.g. 5.9, 6.0 or 6.1, most specifically 6.0, and the formulation further comprises NaCl, preferably at a concentration of 50-100 mM, e.g. 75 mM, and preferably further comprises a non-ionic detergent such as Tween 80, preferably at a concentration of 0.01%.

In accordance with the invention, the formulation can be in liquid, lyophilized, spray dried or frozen form.

Furthermore the invention relates to methods of preparing a formulation according to any aspect as described herein, which may optionally further comprise a step of confectioning the formulation in a dosage unit form.

The invention also provides a method for stabilizing a polypeptide binding to CXCR4, e.g. a polypeptide according to any one of SEQ ID No. 2 to 5, preferably SEQ ID No. 2, for storage, comprising preparing a formulation according to any aspect of the invention.

The invention also provides the use of a formulation according to any aspect as described herein for storage of a polypeptide binding to CXCR4, e.g. a polypeptide according to SEQ ID No. 2 to 5, preferably SEQ ID No. 2, wherein in some embodiments storage is 1-24 months, such as 1, 3, 6, 9, 12 or 24 months, preferably at least 3 months, e.g. at a temperature between −70° C. and +40° C., such as −70° C., −20° C., +5° C., +25° C. or +40° C., preferably a temperature between −70° C. and +25° C.

The invention also provides a pharmaceutical or diagnostic composition comprising a formulation of the polypeptide according to any aspect as describe herein, or obtainable by any method as described herein.

In some embodiments the formulation according to any aspect of the invention is for use in a method of treating a human or animal subject, e.g. for use in treating cancer or AIDS.

The invention relates to a sealed container comprising one or more of the formulations according to any aspect of the invention; or the formulation obtainable by the method according to any aspect of the invention; or a pharmaceutical or diagnostic composition as described herein.

The invention also provides a pharmaceutical unit dosage form suitable for parenteral administration to a patient, preferably a human patient, comprising one or more of the formulations according to any aspect of the invention; or the formulation obtainable by a method as described herein; or the pharmaceutical composition as defined herein; or the sealed container as defined herein.

Finally, the invention provides a kit comprising one or more of the formulations according to any one of the aspects described herein; or the formulation obtainable by a method as described herein; or the pharmaceutical composition as described herein; or the sealed container as described herein; or the pharmaceutical unit dosage form as described herein.

### BRIEF DESCRIPTION OF THE FIGURES

FIG. 1: the sequence of human CXCR4, i.e. GenBank entry: AF005058.1 (SEQ ID NO: 1).

FIG. 2: Format of biparatopic constructs against CXCR4, exemplified by the 4CXCR104 Nanobody. The two different immunoglobulin single variable domains are depicted as separate ovals. For sequence information see also FIG. 3. Concerning the definition of “4CXCR016” and “4CXCR026” see also U.S. Ser. No. 61/358,495. The black line indicates a peptide linker, in particular a 20GS linker as can also be discerned from the sequence of 4CXCR104 in FIG. 3.

FIG. 3: sequences of specific embodiments of biparatopic Nanobody constructs against CXCR4 of the present invention.

FIG. 4: Absorbance values at 280 nm (A) and aggregation indices ([(100×A340)/(A280-A340)]) (B) of 4CXCR104 formulated in buffer 1-6 after 7 weeks storage at +40° C. An overview of the buffer compositions is given in Table 4.

FIG. 5: Kinetics of pyroglutamate formation (A) and degradation (B). Kinetics of pyroglutamate formation upon storage of 4CXCR104 at +40° C. formulated in buffer 1-6. Pyroglutamate formation is significantly slower in citrate buffer (buffer 4-6) compared to phosphate buffer (buffer 1-3). An overview of the buffer compositions is given in Table 4. Kinetics of degradation upon storage of 4CXCR104 at +40° C. formulated in buffer 1-6. Degradation is significantly slower in citrate buffer (buffer 4-6) compared to phosphate buffer (buffer 1-3). An overview of the buffer compositions is given in Table 4.

FIG. 6: Kinetics of proteolytic degradation (A) upon storage of 4CXCR104 at −70±10° C., −20±5° C., +5±3° C., +25±2° C. and +40±2° C. based on RP-HPLC analysis. Kinetics of pyroglutamate formation (B) upon storage of 4CXCR104 at −70° C., −20° C., +5° C., +25° C. and +40° C. based on RP-HPLC analysis.

### DETAILED DESCRIPTION OF THE INVENTION

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.

### Polypeptide of the Invention

The present invention relates to polypeptides comprising at least one immunoglobulin single variable domain directed against a target. Targets in the context of this application include therapeutic targets and targets that can provide half life extension. The latter category includes serum proteins having a long half life, such as serum albumin, immunoglobulins (e.g. IgG) or transferrin, in particular serum albumin. In certain embodiments the invention relates to polypeptides capable of binding to GPCRs, in particular immunoglobulin sequences binding to CXCR4. The terms “polypeptide” and “amino acid sequence” are used interchangeably herein. Thus, an amino acid sequence of the invention is an amino acid sequence capable of binding to a therapeutic target, such as e.g. a GPCR, and in particular CXCR4.

Unless indicated otherwise, the term “immunoglobulin sequence”—whether used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as VHH domains or VH/VL domains, respectively). The terms antigen-binding molecules or antigen-binding protein are used interchangeably with immunoglobulin sequence, and include immunoglobulin single variable domains, such as Nanobodies®. “Nanobody®” and “Nanobodies®” are registered trademarks of Ablynx NV. The terms encompass, inter alia, VHH domains, humanized VHH domains and camelized VH domains, all as previously described.

Embodiments of the invention relate to immunoglobulin sequences that are immunoglobulin single variable domains, such as light chain variable domain sequences (e.g. a VL-sequence), or heavy chain variable domain sequences (e.g. a VH-sequence); more specifically, heavy chain variable domain sequences that are derived from a conventional four-chain antibody or heavy chain variable domain sequences that are derived from a heavy chain antibody.

The term “immunoglobulin single variable domain”, defines molecules wherein the antigen binding site is present on, and formed by, a single immunoglobulin domain or suitable fragments thereof. This sets immunoglobulin single variable domains apart from “conventional” immunoglobulins or their fragments, wherein two immunoglobulin domains, in particular two variable domains interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation.

In contrast, the binding site of an immunoglobulin single variable domain is formed by a single VH or VL domain. Hence, the antigen binding site of an immunoglobulin single variable domain is formed by no more than three CDRs, e.g. one to three CDRs.

The term “immunoglobulin single variable domain” hence does not comprise conventional immunoglobulins or their fragments (such as Fab, Fab2, scFV, diabodies) which require interaction of at least two variable domains for the formation of an antigen binding site. This is also the case for embodiments of the invention which “comprise” or “contain” an immunoglobulin single variable domain. In the context of the present invention, such embodiments exclude conventional immunoglobulins or their fragments. Thus, a composition that “comprises” or “contains” an immunoglobulin single variable domain may relate to e.g. constructs comprising more than one immunoglobulin single variable domain. Alternatively, there may be further constituents other than the immunoglobulin single variable domains, e.g. auxiliary agents of different kinds, protein tags, colorants, dyes, etc. However, these terms do comprise fragments of conventional immunoglobulins wherein the antigen binding site is formed by a single variable domain.

According to the invention, the polypeptide of the invention, more specifically the immunoglobulin sequences, can consist of, or comprise one or more of the following: domain antibodies, or amino acid sequences that are suitable for use as domain antibodies, single domain antibodies, or amino acid sequences that are suitable for use as single domain antibodies, “dAbs”, or amino acid sequences that are suitable for use as dAbs, or Nanobodies®, including but not limited to VHH sequences, and preferably are Nanobodies®.

The present invention encompasses suitable fragments of immunoglobulin single variable domains. “Suitable fragments” of immunoglobulin single variable domains relate to polypeptides which contain fewer amino acids than a native immunoglobulin single variable domain, but still show antigen binding activity (which will then usually contain at least some of the amino acid residues that form at least one of the CDR's, as further described herein). Such single variable domains and fragments most preferably comprise an immunoglobulin fold or are capable for forming, under suitable conditions, an immunoglobulin fold. More specifically, immunoglobulin single variable domains and their fragments are such that are capable of binding to the target antigen. As such, the single variable domain may for example comprise a light chain variable domain sequence (e.g. a V_{L}-sequence) or a suitable fragment thereof; or a heavy chain variable domain sequence (e.g. a V_{H}-sequence or V_{HH} sequence) or a suitable fragment thereof; as long as it is capable of forming a single antigen binding unit (i.e. a functional antigen binding unit that essentially consists of the single variable domain, such that the single antigen binding domain does not need to interact with another variable domain to form a functional antigen binding unit, as is for example the case for the variable domains that are present in for example conventional antibodies and scFv fragments that need to interact with another variable domain—e.g. through a V_{H}/V_{L} interaction—to form a functional antigen binding domain).

The immunoglobulin sequences of the invention are preferably in isolated form or in essentially isolated form, or form part of a protein or polypeptide of the invention (as defined herein), which may comprise or essentially consist of one or more amino acid sequences of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more targets that may be the same or different to the target of the first immunoglobulin single variable domain), so as to provide a monovalent, multivalent or multispecific polypeptide of the invention, respectively, all as described herein. Such a protein or polypeptide may also be in isolated or in essentially isolated form. “Essentially isolated form” means a form where the immunoglobulin single variable domains are the main constituents of a composition, e.g. the main protein constituent, excluding the presence of contaminating substances such as other proteins, residues of host organisms used in production, such as endotoxins, etc., other than in trace amounts. “Essentially isolated” does not exclude the presence of buffers, excipients etc., i.e. substances that are deliberately used for the preparation of a formulation or pharmaceutical composition.

The invention relates to immunoglobulin sequences of different origin, comprising mouse, rat, rabbit, donkey, human and camelid immunoglobulin sequences. The invention also includes fully human, humanized or chimeric immunoglobulin sequences. For example, the invention comprises camelid immunoglobulin sequences and humanized camelid immunoglobulin sequences, or camelized domain antibodies, e.g. camelized Dab as described by Ward et al (see for example WO 94/04678 and Davies and Riechmann (1994 and 1996)). Moreover, the invention comprises fused immunoglobulin sequences, e.g. forming a multivalent and/or multispecific construct (for multivalent and multispecific polypeptides containing one or more VHH domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g. toxins, labels, radiochemicals, etc., which are derivable from the immunoglobulin sequences of the present invention. Immunoglobulin single variable domains have also been described in sharks (also referred to as “IgNARs”, as described e.g. in WO 03/014161 or Streltsov, 2005).

In a particular embodiment, the immunoglobulin single variable domains of the invention are Nanobodies, in particular (camelid) VHH domains, humanized VHH domains or camelized VH domains. The skilled person is well acquainted with humanization of VHH and/or camelizing VH domains.

The amino acid sequence and structure of an immunoglobulin sequence, in particular a Nanobody can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively.

The total number of amino acid residues in a Nanobody® can be in the region of 110-120, is preferably 112-115, and is most preferably 113. It should however be noted that parts, fragments, analogs or derivatives (as further described herein) of a Nanobody® are not particularly limited as to their length and/or size, as long as such parts, fragments, analogs or derivatives meet the further requirements outlined herein and are also preferably suitable for the purposes described herein.

Thus, generally, immunoglobulin single variable domains will be amino acid sequences that consist of, or essentially consist of 4 framework regions (FR1 to FR4 respectively) and 3 complementarity determining regions (CDR1 to CDR3 respectively). “Essentially consist” in this context means that additional elements such as e.g. tags used for purification or labelling may be present, but such additional elements are small as compared to the immunoglobulin single variable domain per se, and do not interfere with the antigen binding activity of the immunoglobulin single variable domain.

As used herein, the term “immunoglobulin sequences” or “immunoglobulin single variable domains” refers to both the nucleic acid sequences coding for the polypeptide, and the polypeptide per se. Any more limiting meaning will be apparent from the particular context.

In particular, the amino acid sequence of the invention may be a Nanobody or a suitable fragment thereof. For a further description of V_{HH}'s and Nanobodies®, reference is made to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference. As described in these references, Nanobodies® (in particular V_{HH} sequences and partially humanized Nanobodies®) can in particular be characterized by the presence of one or more “Hallmark residues” in one or more of the framework sequences. A further description of the Nanobodies®, including humanization and/or camelization of Nanobodies®, as well as other modifications, parts or fragments, derivatives or “Nanobody fusions”, multivalent constructs (including some non-limiting examples of linker sequences) and different modifications to increase the half-life of the Nanobodies® and their preparations can be found e.g. in WO 07/104,529.

According to the invention, immunoglobulin single variable domains comprise constructs comprising two or more antigen binding units in the form of single domains, as outlined above. For example, two (or more) immunoglobulin single variable domains with the same or different antigen specificity can be linked to form e.g. a bivalent, trivalent or multivalent construct. By combining immunoglobulin single variable domains of two or more specificities, bispecific, trispecific etc. constructs can be formed. For example, a polypeptide according to the invention may comprise two immunoglobulin single variable domains directed against target A, and one immunoglobulin single variable domain against target B. Such constructs and modifications thereof, which the skilled person can readily envisage, are all encompassed by the present invention. In particular embodiments, the invention relates to bi-paratopic constructs comprising at least two immunoglobulin single variable domains directed to different epitopes within the same target antigen.

All these molecules are also referred to as “polypeptide of the invention”, which is synonymous with “immunoglobulin sequences” or “immunoglobulin single variable domains” of the invention.

In addition, the term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acid sequences or nucleotide sequences encoding the same, unless the context requires a more limited interpretation.

According to one non-limiting embodiment of the invention, the immunoglobulin sequences, Nanobody® or polypeptide of the invention is glycosylated. According to another non-limiting embodiment of the invention, the immunoglobulin sequences, Nanobody® or polypeptide of the invention is non-glycosylated.

### “Binding” to an Antigen

The invention relates to immunoglobulin sequences that can bind to and/or have affinity for an antigen, e.g. a therapeutic target or a target providing half life extension, such as serum albumin. In particular embodiments, the immunoglobulin sequences bind to either CXCR4 or serum albumin. In the context of the present invention, “binding to and/or having affinity for” a certain antigen has the usual meaning in the art as understood e.g. in the context of antibodies and their respective antigens.

In particular embodiments of the invention, the term “binds to and/or having affinity for” means that the immunoglobulin sequence specifically interacts with an antigen, and is used interchangeably with immunoglobulin sequences “against” the said antigen.

The term “specificity” refers to the number of different types of antigens or antigenic determinants to which a particular immunoglobulin sequence, antigen-binding molecule or antigen-binding protein (such as a Nanobody® or a polypeptide of the invention) can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody® or polypeptide of the invention) and the pertinent antigen. Avidity is related to both the affinity between an antigenic determinant and its antigen binding site on the antigen-binding molecule and the number of pertinent binding sites present on the antigen-binding molecule.

Typically, immunoglobulin sequences of the present invention (such as the amino acid sequences, Nanobodies® and/or polypeptides of the invention) will bind to their antigen with a dissociation constant (KD) of 10⁻⁵ to 10⁻¹² moles/liter or less, and preferably 10⁻⁷ to 10⁻¹2 moles/liter or less and more preferably 10⁻⁸ to 10⁻¹² moles/liter (i.e. with an association constant (KA) of 10⁵ to 10¹² liter/moles or more, and preferably 10⁷ to 10¹² liter/moles or more and more preferably 10⁸ to 10¹² liter/moles),

and/or
bind to cell associated antigens as defined herein with a kon-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
and/or
bind to cell associated antigens as defined herein with a koff rate between 1 s⁻¹ (t1/2=0.69 s) and 10⁻⁶ s⁻¹ (providing a near irreversible complex with a t1/2 of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Any KD value greater than 10⁻⁴ mol/liter (or any KA value lower than 10⁴ M⁻¹) liters/mol is generally considered to indicate non-specific binding.

Preferably, a monovalent immunoglobulin sequence of the invention will bind to the desired antigen with an affinity less than 500 nM, preferably less than 200 nM, more preferably less than 10 nM, such as less than 500 pM.

Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (KA), by means of the relationship [KD=1/KA].

The affinity denotes the strength or stability of a molecular interaction. The affinity is commonly given as by the KD, or dissociation constant, which has units of mol/liter (or M). The affinity can also be expressed as an association constant, KA, which equals 1/KD and has units of (mol/liter)⁻¹ (or M⁻¹). In the present specification, the stability of the interaction between two molecules (such as an amino acid sequence, immunoglobulin sequence, Nanobody® or polypeptide of the invention and its intended target) will mainly be expressed in terms of the KD value of their interaction; it being clear to the skilled person that in view of the relation KA=1/KD, specifying the strength of molecular interaction by its KD value can also be used to calculate the corresponding KA value. The KD-value characterizes the strength of a molecular interaction also in a thermodynamic sense as it is related to the free energy (DG) of binding by the well known relation DG=RT.ln(KD) (equivalently DG=−RT.ln(KA)), where R equals the gas constant, T equals the absolute temperature and In denotes the natural logarithm.

The KD for biological interactions, such as the binding of the immunoglobulin sequences of the invention to the cell associated antigen as defined herein, which are considered meaningful (e.g. specific) are typically in the range of 10⁻¹⁰M (0.1 nM) to 10⁻⁵M (10000 nM). The stronger an interaction is, the lower is its KD.

The KD can also be expressed as the ratio of the dissociation rate constant of a complex, denoted as koff, to the rate of its association, denoted kon (so that KD=koff/kon and KA=kon/koff). The off-rate koff has units s⁻¹ (where s is the SI unit notation of second). The on-rate kon has units M⁻¹s⁻¹.

As regards immunoglobulin sequences of the invention, the on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t1/2=ln(2)/koff. The off-rate of immunoglobulin sequences of the invention may vary between 10⁻⁶ s⁻¹ (near irreversible complex with a t1/2 of multiple days) to 1 s⁻¹ (t1/2=0.69 s).

The affinity of a molecular interaction between two molecules can be measured via different techniques known per se, such as the well known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kon, koff measurements and hence KD (or KA) values. This can for example be performed using the well-known Biacore instruments.

It will also be clear to the skilled person that the measured KD may correspond to the apparent KD if the measuring process somehow influences the intrinsic binding affinity of the implied molecules for example by artefacts related to the coating on the biosensor of one molecule. Also, an apparent KD may be measured if one molecule contains more than one recognition sites for the other molecule. In such situation the measured affinity may be affected by the avidity of the interaction by the two molecules.

Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artefacts relating to adsorption of one of the molecules on a support such as plastic.

However, the accurate measurement of KD may be quite labour-intensive and as consequence, often apparent KD values are determined to assess the binding strength of two molecules. It should be noted that as long as all measurements are made in a consistent way (e.g. keeping the assay conditions unchanged) apparent KD measurements can be used as an approximation of the true KD and hence in the present document KD and apparent KD should be treated with equal importance or relevance.

Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labelled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of A is varied for a given concentration or amount of B. As a result an IC50 value is obtained corresponding to the concentration of A at which the signal measured for C in absence of A is halved. Provided KD ref, the KD of the reference molecule, is known, as well as the total concentration cref of the reference molecule, the apparent KD for the interaction A-B can be obtained from following formula: KD=IC50/(1+cref/KD ref). Note that if cref<<KD ref, KD≈IC50. Provided the measurement of the IC50 is performed in a consistent way (e.g. keeping cref fixed) for the binders that are compared, the strength or stability of a molecular interaction can be assessed by the IC50 and this measurement is judged as equivalent to KD or to apparent KD throughout this text.

### Target Antigen

The immunoglobulin single variable domains of the present invention bind to and/or have affinity to target antigens. Target antigens include therapeutic targets as well as targets that provide for half life extension, e.g. long lived serum components such as serum albumin, immunoglobulins (in particular IgG) and transferrin. In particular embodiments, the target antigen is a GPCR, in particular CXC receptors, such as CXCR4, or serum albumin (including human serum albumin, rat serum albumin, mouse serum albumin, rabbit serum albumin or monkey serum albumin).

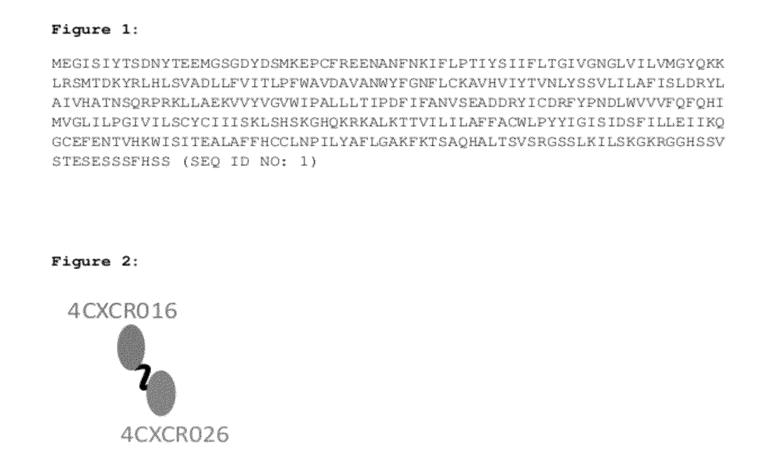

In the context of the present invention, “CXCR4” includes, but is not limited to mouse, and/or human CXCR4 and most preferred human CXCR4, i.e. GenBank entry: AF005058.1 (SEQ ID NO: 1: MEGISIYTSDNYTEEMGSGDYDSMKEPCFREENANFNKIFLPTIYSIIFLTGIVGNGLVILVMGYQKK LRSMTDKYRLHLSVADLLFVITLPFWAVDAVANWYFGNFLCKAVHVIYTVNLYSSVLILAFISLDRYL AIVHATNSQRPRKLLAEKVVYVGVWIPALLLTIPDFIFANVSEADDRYICDRFYPNDLWVVVFQFQHI MVGLILPGIVILSCYCIIISKLSHSKGHQKRKALKTTVILILAFFACWLPYYIGISIDSFILLEIIKQ GCEFENTVHKWISITEALAFFHCCLNPILYAFLGAKFKTSAQHALTSVSRGSSLKILSKGKRGGHSSV STESESSSFHSS, see FIG. 1)

The skilled person is well acquainted with suitable techniques for generating polypeptide sequences, e.g. immunoglobulin single variable domains, against a target antigen. Suitable techniques are described e.g. in copending PCT application WO 09/138,519, copending application U.S. Ser. No. 61/358,495, copending PCT application claiming priority of U.S. 61/293,279, filed on Jan. 7, 2011 with the application number PCT/EP2011/050157, as well as copending PCT application claiming priority of U.S. 61/293,279, filed on Jan. 7, 2011 with the application number PCT/EP2011/050156, all in the name of the present applicant.

### Specific Exemplary Embodiments of Immunoglobulin Sequences

The present invention relates to immunoglobulin single variable domains described in, or obtainable by the methods as disclosed in copending PCT application WO 09/138,519, copending application U.S. Ser. No. 61/358,495, copending PCT application claiming priority of U.S. 61/293,279, filed on Jan. 7, 2011 with the application number PCT/EP2011/050157, as well as copending PCT application claiming priority of U.S. 61/293,279, filed on Jan. 7, 2011 with the application number PCT/EP2011/050156, all in the name of the present applicant.

The international application WO 09/138,519 by Ablynx N.V. entitled “Amino acid sequences directed against CXCR4 and other GPCRs and compounds comprising the same” describes amino acid sequences against G-protein coupled receptors (GPCRs) and in particular human CXCR4, Genbank accession number AF005058.

WO 09/138,519 describes a number of amino acid sequences and in particular VHHs and constructs thereof that are directed against human CXCR4 (see for example the amino acid sequences mentioned such as SEQ ID NO: 238 and SEQ ID NO: 239 in Table B-1.1 of WO 09/138,519). WO 09/138,519 also describes multivalent, multispecific and/or biparatopic constructs (as defined in WO 09/138,519) that are directed against human CXCR4. Reference is for example made to the constructs referred to in Example 4 of WO 09/138,519 such as SEQ ID NO: 264 in Table B-5 of WO 09/138,519), all of which are envisaged as immunoglobulin single variable domains of the present invention.

One particularly preferred example of an amino acid sequence against human CXCR4 from WO 09/138,519 is the sequence called 238D2 (see SEQ ID NO: 238 in WO 09/138,519):

(SEQ ID NO: 6)
EVQLVESGGGLVQTGGSLRLSCAASGFTFSSYAMSWVRQAPGKGLEWV
SGIKSSGDSTRYAGSVKGRFTISRDNAKNMLYLQMYSLKPEDTAVYYC
AKSRVSRTGLYTYDNRGQGTQVTVSS.

One other particularly preferred example of an amino acid sequence against the human CXCR4 from WO 09/138,519 is the sequence called 238D4 (see SEQ ID NO: 239 in WO 09/138,519):

(SEQ ID NO: 7)
EVQLMESGGGLVQAGGSLRLSCAASGRTFNNYAMGWFRRAPGKEREF
VAAITRSGVRSGVSAIYGDSVKDRFTISRDNAKNTLYLQMNSLKPED
TAVYTCAASAIGSGALRRFEYDYSGQGTQVTVSS.

WO 09/138,519 further gives some non-limiting examples of multivalent, multispecific and/or biparatopic constructs that comprise 238D2 and/or 238D4 (see for example SEQ ID NO's: 261 to 266 in WO 09/138,519 and in particular 238D2-20GS-238D4), all of which are envisaged as immunoglobulin single variable domains of the present invention. One other particularly preferred example of an amino acid sequence against the human CXCR4 from WO 09/138,519 is the sequence called 238D2-20GS-238D4 (see SEQ ID NO: 264 in WO 09/138,519):

(SEQ ID NO: 8)
EVQLVESGGGLVQTGGSLRLSCAASGFTFSSYAMSWVRQAPGKGLEWV
SGIKSSGDSTRYAGSVKGRFTISRDNAKNMLYLQMYSLKPEDTAVYYC
AKSRVSRTGLYTYDNRGQGTQVTVSSGGGGSGGGGSGGGGSGGGGSEV
QLMESGGGLVQAGGSLRLSCAASGRTFNNYAMGWFRRAPGKEREFVAA
ITRSGVRSGVSAIYGDSVKDRFTISRDNAKNTLYLQMNSLKPEDTAVY
TCAASAIGSGALRRFEYDYSGQGTQVTVSS.

In particularly preferred embodiments the present invention relates to humanized variants of SEQ ID No. 8, which share at least 90, 95 or 98% identity with SEQ ID No. 8, and preferably have no changes in the CDRs as compared to SEQ ID No. 8.

The non-prepublished U.S. application 61/358,495 by applicant filed on Jun. 25, 2010 entitled “Improved immunoglobulin single variable domains and constructs thereof directed against CXCR-4” describes a number of sequence-optimized/improved variants of the amino acid sequences 238D2 and 238D4, as well as multivalent, multispecific and/or biparatopic constructs that comprise such improved variants as building blocks, all of which are envisaged as immunoglobulin single variable domains of the present invention.

The PCT application PCT/EP2010/064766 by Ablynx N.V. filed on Oct. 4, 2010 and entitled “Immunoglobulin single variable domains directed against human CXCR-4 and other cell-associated proteins and methods to generate them” describes the binding epitope of amino acid sequences 238D2 and 238D4 as well as a number of further immunoglobulin single variable domains capable of binding to the same epitope, all of which are envisaged as immunoglobulin single variable domains of the present invention.

Copending PCT application claiming priority of U.S. 61/293,279, filed on Jan. 7, 2011 with the application number PCT/EP2011/050156, describes further examples of immunoglobulin single variable domains against CXCR4 with improved potency and constructs comprising the same, e.g. in FIGS. 1 to 3, Table B-1 and the specification, claims and figures in general. This application describes monovalent binders, biparatopic binders, and half-life extended binders, all of which are envisaged as immunoglobulin single variable domains of the present invention.

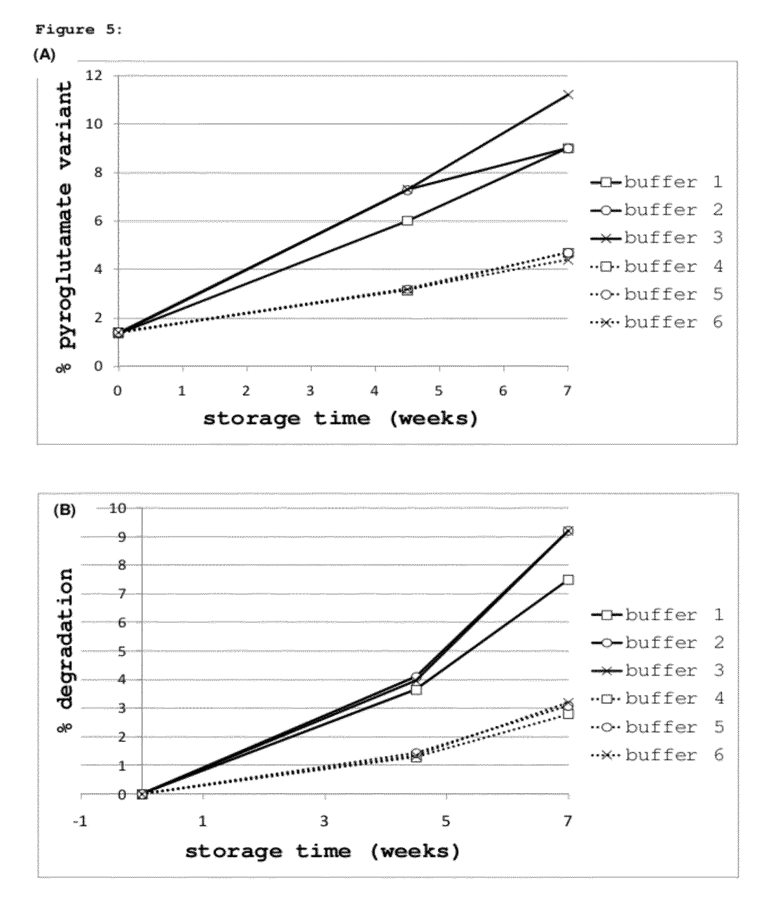

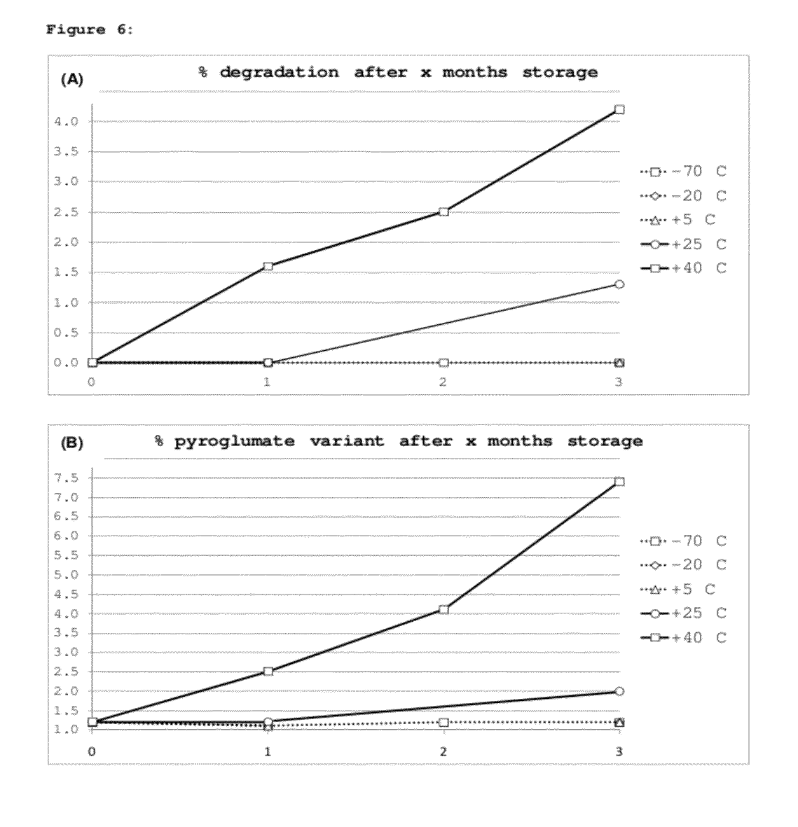

Copending PCT application claiming priority of U.S. 61/293,279, filed on Jan. 7, 2011 with the application number PCT/EP2011/050157, describes further examples of immunoglobulin single variable domains directed against CXCR4 as well as methods of creating such immunoglobulin single variable domains, see e.g. FIG. 5D, Table A-1 and Table A-2, all of which are envisaged as immunoglobulin single variable domains of the present invention.

A preferred, but non-limiting embodiment of an amino acid sequence of the invention is the amino acid sequence referred to as 4CXCR104 (SEQ ID NO: 2, see also SEQ ID No. 7 of U.S. Ser. No. 61/358,495):

(SEQ ID NO: 2 or 4CXCR104)
EVQLVESGGGLVQPGGSLRLSCAASGFTFSSYAMSWVRQAPGKGLEW
VSGIKSSGDSTRYAGSVKGRFTISRDNAKNTLYLQMNSLRPEDTAVY
YCAKSRVSRTGLYTYDNRGQGTLVTVSSGGGGSGGGGSGGGGSGGGG
SEVQLVESGGGLVQPGGSLRLSCAASGRTFNNYAMGWFRQAPGKERE
FVAAITRSGVRSGVSAIYGDSVKDRFTISRDNAKNTLYLQMNSLRPE
DTAVYYCAASAIGSGALRRFEYDYSGQGTLVTVSS.

As compared to the amino acid sequence defined as “238D2-20GS-238D4” in WO 09/138,519 the above sequence comprises the following amino acid changes: for the 238D2 building block: T14P, M77T, Y82aN, K83R, and Q108L and for the 238D4 building block: M5V, A14P, R39Q, K83R, T91Y, and Q108L.

In a further embodiment, the amino acid sequence of the invention is a variant of 238D2-20GS-238D4 (as defined in WO 09/138,519) that comprises, at position 5 of the 238D4 building block a valine instead of the original methionine residue. In this aspect, the amino acid sequence of the invention is also referred to as 4CXCR100 (SEQ ID NO: 3; see also SEQ ID No. 4 in U.S. Ser. No. 61/358,495):

(SEQ ID NO: 3 or 4CXCR100)
EVQLVESGGGLVQTGGSLRLSCAASGFTFSSYAMSWVRQAPGKGLEW
VSGIKSSGDSTRYAGSVKGRFTISRDNAKNMLYLQMYSLKPEDTAVY
YCAKSRVSRTGLYTYDNRGQGTQVTVSSGGGGSGGGGSGGGGSGGGG
SEVQLVESGGGLVQAGGSLRLSCAASGRTFNNYAMGWFRRAPGKERE
FVAAITRSGVRSGVSAIYGDSVKDRFTISRDNAKNTLYLQMNSLKPE
DTAVYTCAASAIGSGALRRFEYDYSGQGTQVTVSS.

In another embodiment, the amino acid sequence of the invention is a variant of 238D2-20GS-238D4 (as defined in WO 09/138,519) that comprises, in addition to the amino acid exchange of SEQ ID No. 2, at position 77 of the 238D2 building block a threonine instead of the original methionine residue. In this aspect, the amino acid sequence of the invention is also referred to as 4CXCR101 (SEQ ID NO: 4, see also Seq. Id. No.: 5 in U.S. Ser. No. 61/358,495):

(SEQ ID NO: 4 or 4CXCR101)
EVQLVESGGGLVQTGGSLRLSCAASGFTFSSYAMSWVRQAPGKGLEW
VSGIKSSGDSTRYAGSVKGRFTISRDNAKNTLYLQMYSLKPEDTAVY
YCAKSRVSRTGLYTYDNRGQGTQVTVSSGGGGSGGGGSGGGGSGGGG
SEVQLVESGGGLVQAGGSLRLSCAASGRTFNNYAMGWFRRAPGKERE
FVAAITRSGVRSGVSAIYGDSVKDRFTISRDNAKNTLYLQMNSLKPE
DTAVYTCAASAIGSGALRRFEYDYSGQGTQVTVSS.

In a further embodiment of the invention, the amino acid sequences contains compared to the sequence 238D2-20GS-238D4 (as defined in WO 09/138,519) at least the substitutions: for the 238D2 building block: T14P, M77T, Y82aN, K83R, and Q108L and for the 238D4 building block: M5V; also referred to herein as 4CXCR103 (SEQ ID NO: 5, see also Seq. Id. No.: 6 in U.S. Ser. No. 61/358,495):

(SEQ ID NO: 5 or 4CXCR103)
EVQLVESGGGLVQPGGSLRLSCAASGFTFSSYAMSWVRQAPGKGLEW
VSGIKSSGDSTRYAGSVKGRFTISRDNAKNTLYLQMNSLRPEDTAVY
YCAKSRVSRTGLYTYDNRGQGTLVTVSSGGGGSGGGGSGGGGSGGGG
SEVQLVESGGGLVQAGGSLRLSCAASGRTFNNYAMGWFRRAPGKERE
FVAAITRSGVRSGVSAIYGDSVKDRFTISRDNAKNTLYLQMNSLKPE
DTAVYTCAASAIGSGALRRFEYDYSGQGTQVTVSS.

The invention also encompasses optimized variants of these amino acid sequences. Generally, an “optimized variant” of an amino acid sequence according to the invention is a variant that comprises one or more beneficial substitutions such as a substitutions increasing i) the degree of “humanization”, ii) the chemical stability, and/or iii) the level of expression; while the potency (measured e.g. by the potency assay as described in the experimental part of WO 09/138,519 or in U.S. Ser. No. 61/358,495) remains comparable (i.e. within a 10% deviation) to the wild type 238D2-20GS-238D4 (as defined in WO 09/138,519) or comparable to the variant 4CXCR100 (SEQ ID NO: 5). Preferably, compared to the wild-type sequence of 238D2-20GS-238D4 (SEQ ID No. 8), an amino acid sequence of the invention contains at least one such substitution, and preferably at least two such substitutions, and preferably at least three humanizing substitutions and preferably at least 10 such humanizing substitutions. Also, again compared to the wild-type sequence 238D2-20GS-238D4, the amino acid sequences of the invention preferably comprise a maximum of 20 substitutions, and preferably a total of 15, 13, 11 or 10 substitutions. Some preferred, but non-limiting examples of such substitutions include for the 238D2 building block: T14P, M77T, Y82aN, K83R, and/or Q108L; and for the 238D4 building block: M5V, A14P, R39Q, K83R, T91Y, and/or Q108L (numbering according to FIG. 5 of U.S. Ser. No. 61/358,495, see also SEQ ID No. 6 and 7, respectively).

In a particular aspect, the amino acid sequences of the invention contain a total of between 6 and 15, preferably between 9 and 13, such as 10, 11 or 12 amino acid substitutions compared to the wild-type sequence 238D2-20GS-238D4. As mentioned, these differences preferably at least comprise one and preferably both of the substitutions M5V in the 238D4 building block and/or M77T in the 238D2 building block, and at least one, preferably at least two, such as three, four or five or ten humanizing substitutions, and may optionally comprise one or more further substitutions (such as any one of, or any suitable combination of any two or more of, the further substitutions (a) to (c) as mentioned herein). Again, based on the disclosure herein and optionally after a limited degree of trial and error, the skilled person will be able to select (a suitable combination of) one or more such suitable humanizing and/or further substitutions.

The present invention encompasses polypeptide sequences that are highly similar to any of the specific examples provided herein, or any of the specific examples defined by reference above. Highly similar means an amino acid identity of at least 90%, e.g. 95, 97, 98 or 99%. The highly similar polypeptide sequences will have the same function as the sequence they are derived from, i.e. they will e.g. bind to CXCR4, more specifically bind to and inhibit signalling via this receptor. In preferred embodiments the CDRs remain unchanged as compared to SEQ ID No. 8.

In a particular embodiment, the invention relates to sequences highly similar to any one of SEQ ID No. 2 to 5, in particular SEQ ID No. 2 (wherein preferably the CDRs remain unchanged). The invention in particular refers to variants or highly similar sequences which are stable in the formulations as defined herein.

Methods to generate polypeptide sequences of the invention are widely known and include e.g. recombinant expression or synthesis. The skilled person is well acquainted with suitable expression technology, e.g. suitable recombinant vectors and host cells, e.g. bacterial or yeast host cells. The skilled person is also well acquainted with suitable purification techniques and protocols.

### Half Life Extending Amino Acid Sequences

In particular embodiments the present invention relates to formulations of immunoglobulin single variable domains that can bind to a target providing half life extension, such as serum proteins having a long half life, e.g. serum albumin, immunoglobulins, or transferrin, preferably serum albumin, in particular human serum albumin. Half life preferably relates to T1/2β of an immunoglobulin single variable domain or construct. In the context of the present invention, the “immunoglobulin single variable domains that can bind to a target providing half life extension”, in particular the “serum-albumin binding polypeptide or binding domain” may be any suitable serum-albumin binding peptide or binding domain capable of increasing the half-life (preferably T1/2β) of the construct (compared to the same construct without the serum-albumin binding peptide or binding domain).

Polypeptide sequences capable of binding to serum albumin have previously been described and may in particular be serum albumin binding peptides as described in WO 2008/068280 by applicant (and in particular WO 2009/127691 and the non-prepublished U.S. application 61/301,819, both by applicant), or a serum-albumin binding immunoglobulin single variable domains (such as a serum-albumin binding Nanobody; for example Alb-1 or a humanized version of Alb-1 such as Alb-8, for which reference is for example made to WO 06/122787).

Further specific examples of such binders are known in the art. For example, the present invention relates to formulations of binders described in WO2006/122787, in particular humanized variants of the sequence PMP6A6 according to SEQ ID NO: 52, more in particular amino acid sequences chosen from the group comprising the clones ALB 3 according to SEQ ID NO: 57; ALB 4 according to SEQ ID NO: 58; ALB 5 according to SEQ ID NO: 59; ALB 6 according to SEQ ID NO: 60; ALB 7 according to SEQ ID NO: 61; ALB 8 according to SEQ ID NO: 62; ALB 9 according to SEQ ID NO: 63; and ALB 10 according to SEQ ID NO: 64, preferably ALB 8 according to SEQ ID NO: 62, each sequence as described in WO 2006/122787. In particular embodiments the present invention relates to any of these sequences without a tag, such as a His tag or a Flag tag. Tag sequences are well known to the skilled person, such that they can be readily determined and removed in any of the specific embodiments referred to herein.

The present invention relates to formulations comprising any one or more of these amino acid sequences, in particular the humanized variants of PMP6A6, more in particular Alb8 (or a tag-less version thereof) as defined above, wherein the formulations are characterized as further described herein. The present invention advantageously provides stable formulations of these amino acid sequences.

Moreover, as detailed above, the present invention pertains to immunoglobulin single variable domains and constructs comprising one or more immunoglobulin single variable domains, e.g. monovalent, bivalent, multivalent, biparatopic constructs comprising any one or more of the above half life extending sequences.

It is thus also envisioned in the context of the present invention to use constructs comprising one or more immunoglobulin single variable domains against a therapeutic target in combination with one or more immunoglobulin single variable domain providing half life extension as defined herein, (preferably T1/2β) of the construct. In these constructs, the “serum-albumin binding polypeptide or binding domain” may be any suitable serum-albumin binding peptide or binding domain capable of increasing the half-life (preferably T1/2β) of the construct (compared to the same construct without the serum-albumin binding peptide or binding domain). Specifically, the polypeptide sequence suitable for extending serum half life is a polypeptide sequence capable of binding to a serum protein with a long serum half life, such as serum albumin, transferrin, IgG, etc, in particular serum albumin. Polypeptide sequences capable of binding to serum albumin have previously been described and may in particular be serum albumin binding peptides as described in WO 2008/068280 by applicant (and in particular WO 2009/127691 and the non-prepublished U.S. application 61/301,819, both by applicant), or a serum-albumin binding immunoglobulin single variable domains (such as a serum-albumin binding Nanobody; for example Alb-1 or a humanized version of Alb-1 such as Alb-8, for which reference is for example made to WO 06/122787).

More in particular one or more immunoglobulin single variable domains that are humanized variants of the sequence PMP6A6 according to SEQ ID NO: 52, more in particular amino acid sequences chosen from the group comprising the clones ALB 3 according to SEQ ID NO: 57; ALB 4 according to SEQ ID NO: 58; ALB 5 according to SEQ ID NO: 59; ALB 6 according to SEQ ID NO: 60; ALB 7 according to SEQ ID NO: 61; ALB 8 according to SEQ ID NO: 62; ALB 9 according to SEQ ID NO: 63; and ALB 10 according to SEQ ID NO: 64, preferably ALB 8 according to SEQ ID NO: 62, each as described in WO2006/122787, or a tag-less versions of any one of these.

In particular embodiments the present invention relates to immunoglobulin single variable domains binding to CXCR4, e.g. according to SEQ ID No. 2-5, in particular SEQ ID No. 2, in combination with at least one polypeptide sequence or binding domain suitable for extending serum half life. In particular the binding domain suitable for extending serum half life that is combined in a construct comprising one or more of SEQ ID No. 2-5 (including constructs having two or three binders of the same sequence) can be a polypeptide sequences capable of binding to serum albumin as previously described and may in particular be serum albumin binding peptides as described in WO 2008/068280 by applicant (and in particular WO 2009/127691 and the non-prepublished U.S. application 61/301,819, both by applicant), or a serum-albumin binding immunoglobulin single variable domains (such as a serum-albumin binding Nanobody; for example Alb-1 or a humanized version of Alb-1 such as Alb-8, for which reference is for example made to WO 06/122787).

More in particular, the serum albumin binding domain may be one or more immunoglobulin single variable domain selected from humanized variants of the sequence PMP6A6 according to SEQ ID NO: 52, more in particular amino acid sequences chosen from the group comprising the clones ALB 3 according to SEQ ID NO: 57; ALB 4 according to SEQ ID NO: 58; ALB 5 according to SEQ ID NO: 59; ALB 6 according to SEQ ID NO: 60; ALB 7 according to SEQ ID NO: 61; ALB 8 according to SEQ ID NO: 62; ALB 9 according to SEQ ID NO: 63; and ALB 10 according to SEQ ID NO: 64, preferably ALB 8 according to SEQ ID NO: 62, each as described in WO 2006/122787, or a tag-less version of any one of these.

The formulations of the present invention have the particular advantage that they prevent or reduce oligomerization, in particular dimerization of the above binders. More in particular, the formulations based on citrate buffer as described herein serve to have this beneficial effect.

In constructs according to the present invention the immunoglobulin single variable domains binding to CXCR4 and polypeptide sequences or domains suitable for extending serum half life can be fused with or without a linker, e.g. a peptide linker. Widely used peptide linkers comprise Gly-Ser repeats, e.g. (Gly)4-Ser in one, two, three, four, five, six or more repeats, e.g. 20 repeats.

When a construct comprising more than one immunoglobulin single variable domain of the present invention, e.g. a bivalent, e.g. biparatopic construct, further comprises a polypeptide sequence for extending half life, as defined herein, the invention encompasses different formats of combining the domains and the polypeptide for extending half life. For example, two target specific immunoglobulin single variable domains (e.g. linked via a linker, e.g. a peptide linker) may be followed, in sequence, by the polypeptide for extending serum half life (e.g. linked via a linker, e.g. a peptide linker). Alternatively, the polypeptide for extending serum half life may be placed in between the two target binding immunoglobulin single variable domains, each in turn optionally linked via a linker, e.g. a peptide linker.

Alternative means for extending half life which are also encompassed by the present invention include e.g. pegylation as widely known in the art, including site specific or random pegylation, preferably site specific pegylation. PEG can be used with a molecular weight above 5000, e.g. between 10.000 and 200.000, preferably in the range between 20.000 and 100.000.

In any aspect of half-life extension, it is envisaged that the activity of the polypeptide as defined herein is not compromised, i.e. it is e.g. at least 75%, 80%, 85%, 90% or 95% of the activity of the same polypeptide without half-life extension. Activity can relate to e.g. binding to the target antigen, and/or potency in a bioassay. The skilled person will also ascertain that the chosen half-life extension technology is suitable in that it does not increase, or even decreases immunogenicity.

### Formulations of the Invention

The present invention provides formulations of polypeptides as defined herein, such as immunoglobulin single variable domains directed against therapeutic targets and/or targets providing for half life extension. In particular embodiments of the invention, the immunoglobulin single variable domains are directed against GPCRs and/or targets that provide half life extension. More in particular, the immunoglobulin single variable domains of the present invention are directed against CXCR4 and/or serum albumin. The invention e.g. relates to immunoglobulin single variable domains or constructs comprising at least one immunoglobulin single variable domain, which are stable, and preferably suitable for pharmaceutical uses, comprising the preparation of medicaments.

More specifically, the formulations of the present invention relate to one or more of the immunoglobulin single variable domains selected from SEQ ID No. 2 to 5, in particular SEQ ID No. 2, or Alb1 to Alb8 (or tag-less versions thereof), or constructs comprising one or more of these immunoglobulin single variable domains. In a more preferred embodiment, the formulations of the present invention relate to SEQ ID No. 2 or constructs comprising one or more such immunoglobulin single variable domain.

Accordingly, the present invention provides formulations characterized by a suitable degree of purity and at suitable concentrations as required e.g. for pharmaceutical purposes. The formulations provide the polypeptides, e.g. immunoglobulin single variable domains or constructs comprising at least one immunoglobulin single variable domain as defined herein in a stable form (as defined herein) over a large range of concentrations, and a large range of storage conditions, e.g. temperatures, including stressed conditions such as elevated temperatures (e.g. 25° C. or higher), shaking or other forms of physical stress.

The formulation comprises an aqueous carrier. The aqueous carrier is in particular a buffer.

The invention, however, also encompasses products obtainable by further processing of a liquid formulation, such as a frozen, lyophilized or spray dried product. Upon reconstitution, these solid products can become liquid formulations as described herein (but are not limited thereto). In its broadest sense, therefore, the term “formulation” encompasses both liquid and solid formulations. However, solid formulations are understood as derivable from the liquid formulations (e.g. by freezing, freeze-drying or spray-drying), and hence have characteristics that are defined by the features specified for liquid formulations herein. The invention does not exclude reconstitution that leads to a composition that deviates from the original composition, i.e. the composition before e.g. freeze- or spray drying.

The formulations of the invention comprise at least one polypeptide sequence, in particular immunoglobulin single variable domains or construct comprising at least one immunoglobulin single variable domain as defined herein. In particular embodiments, the formulation comprises one or more polypeptides selected from SEQ ID No. 2 to SEQ ID No. 5, preferably SEQ ID No. 2. The polypeptides may in addition be half-life extended e.g. by incorporating a serum-albumin binding peptide or binding domain, which may be any suitable serum-albumin binding peptide or binding domain capable of increasing the half-life of the construct (compared to the same construct without the serum-albumin binding peptide or binding domain), and may in particular be serum albumin binding peptides as described in WO 2008/068280 by applicant (and in particular WO 2009/127691 and the non-prepublished U.S. application 61/301,819, both by applicant), or a serum-albumin binding immunoglobulin single variable domain (such as a serum-albumin binding Nanobody; for example Alb-1 or a humanized version of Alb-1 such as Alb-8, for which reference is for example made to WO 06/122787), and more in particular the specific examples of sequences described in WO 06/122787 as referenced hereinabove.

The formulation of the invention comprises a buffer selected from at least one of citrate or phosphate buffer, preferably a citrate buffer. In a particular embodiment, the citrate buffer is prepared using citric acid monohydrate and tri-sodium citrate dehydrate, e.g. 1.325 g/L citric acid monohydrate and 12.850 g/L tri-sodium citrate dehydrate.

The formulation according to the invention comprises the buffer at a concentration in the range of 5-100 mM, e.g. 5, 10, 15, 20, 25, 30, 40, 50, 60, 70, 80, 90 or 100 mM, preferably 5-70 mM, more preferably 5-50 mM, e.g. 10, 20, 30, 40 or 50 mM, wherein each value is understood to optionally encompass a range of ±5 mM.

The pH of the formulation of the invention is in the range 5.0 to 7.5, wherein each value is understood to encompass a range of ±0.2. Specific examples of preferred pH values for formulations of the invention can be selected from the non-limiting list comprising pH of 5.0, 5.5, 6.0, 6.5, 7.0 or 7.5, preferably 5.5 to 6.5, more preferably 5.9, 6.0, 6.1, e.g. 6.0 wherein each value is understood to optionally encompass a range of ±0.2.

The most advantageous pH will depend on the buffer comprised in the formulation. Hence the invention relates to e.g. a formulation selected from a) or b)
a) a formulation comprising a phosphate buffer, which preferably has a pH in the range of 6.5 to 7.5, e.g. 6.5 to 7.0, preferably 6.9, 7.0 7.1, e.g. 7.0 wherein each value is understood to optionally encompass a range of ±0.2; or
b) a formulation comprising a citrate buffer, which preferably has a pH between 5.5 and 6.5, more preferably 5.9, 6.0, 6.1, e.g. 6.0 wherein each value is understood to optionally encompass a range of ±0.2. In one preferable embodiment the formulation is a formulation according to b).

The formulations of the invention will comprise the polypeptides as defined herein, in particular the immunoglobulin single variable domains or constructs comprising at least one immunoglobulin single variable domain at a concentration that is suitable for clinical purposes, which includes concentrations used in stock solutions for dilution prior to use on the patient.

Typical concentrations of the active agent in formulations of the invention comprise the non-limiting examples of concentrations in the range of 0.1 to 150 mg/ml, preferably 5-50 mg/ml, such as 5, 10, 15, 20, 25, 30, 35, 40, 45 or 50 mg/ml, preferably 10 mg/ml, wherein each value is understood to optionally encompass a range of ±20% (e.g. a value of 10 optionally encompasses a range of 8 to 12 mg/ml).

The formulations according to the invention may also optionally comprise one or more excipients. The skilled person is familiar with excipients suitable for pharmaceutical purposes, which may have particular functions in the formulation, such as lyoprotection, stabilization, preservation, etc. Commonly used stabilizers and preservatives are well known to the skilled person (see e.g. WO 2010/077422). In advantageous embodiments, the excipient may be one or more selected from the list consisting of NaCl, sucrose or mannitol.

The skilled person can readily determine suitable concentrations of the excipients to be added to the formulations. In exemplary embodiments, NaCl has a concentration in the range of 10-500 mM, such as 25, 30, 40, 50, 60, 70, 100, 150, 250 or 500 mM, preferably 50-150 mM, e.g. 75 or 100 mM, wherein each value is understood to optionally encompass a range of ±5 mM; and/or mannitol has a concentration of 1-10%, preferably 2-4%, e.g. 2, 3 or 4% (w/w), wherein each value is understood to optionally encompass a range of ±0.5%; and/or sucrose has a concentration of 1-12%, preferably 2-7%, e.g. 4, 5 or 6% (w/w) wherein each value is understood to optionally encompass a range of ±1%.

In a preferred embodiment, the formulations according to any aspect of the invention are isotonic in relation to human blood. Tonicity can be expressed in terms of osmolality, which can be a theoretical osmolality, or preferably an experimentally determined osmolality. Typically, osmolality will be in the range of 290±60 mOsm/kg, preferably 290±20 mOsm/kg.

Thus, in the selection of excipients (if any) the skilled person will consider buffer concentration and the concentrations of the one or more excipients and preferably arrive at a formulation with an osmolality in the ranges as specified above. The skilled person is familiar with calculations to estimate osmolality (see e.g. WO2010/077422). If required, the skilled person can also further include a compound to adjust the osmolality of the formulation. Exemplary compounds include, but are not limited to the above mentioned excipients, and/or one or more of sorbitol, glycine, methionine, dextrose, inositol, arginine, or arginine hydrochloride.

The formulations of the invention may also comprise compounds that are specifically useful for protecting the polypeptide of the invention during freeze-drying. Such compounds are also known as lyoprotectants, and are well known to the skilled person. Specific examples include, but are not limited to sugars like sucrose, sorbitol or threhalose; amino acids such as glutamate, in particular monosodium glutamate or histidine; betain, magnesium sulfate, sugar alcohols, propylene glycol, polyethylene glycols and combinations thereof. The required amount of such a compound to be added can readily be determined by the skilled person under consideration of stability of the formulation in liquid form and when undergoing lyophilization. Formulations that are particularly suitable for freeze-drying may furthermore comprise bulking agents. Suitable agents are widely known to the skilled person.

In a further embodiment of the invention, the formulation according to any aspect of the invention may further comprise a detergent, e.g. a detergent selected from the non-limiting list of polysorbates e.g. polysorbate-20, -40, -60, -65, -80 or -85. The skilled person knows further non-limiting examples of detergents, such as those listed e.g. in WO 2010/077422. In a preferred embodiment, the detergent is a non-ionic detergent. More specifically, the detergent can be Tween-80 (polysorbate-80). The skilled person can readily determine a suitable concentration of detergent for a formulation of the invention. Typically, the concentration will be as low as possible, whilst maintaining the beneficial effects of the detergents, e.g. a stabilizing effect under conditions of shear stress, e.g. stirring. In exemplary, non-limiting embodiments, the concentration of the detergent may be in the range of 0.001 to 0.5%, e.g. 0.001, 0.002, 0.003, 0.004, 0.005, 0.01, 0.015, 0.02, 0.025, 0.03, 0.035, 0.04, 0.045, 0.05%, 0.06%, 0.08% or 0.1%, preferably in a concentration between 0.01 and 0.05%, more preferably between 0.01 and 0.02%, e.g. 0.01% (w/w).

The various embodiments as described above can be combined in formulations of the invention without limitations. However, preferable non-limiting examples of formulations include formulations wherein the buffer is a citrate buffer at pH 6.0, and the formulation further comprises NaCl, preferably at a concentration of 75 mM, and preferably further comprises a non-ionic detergent such as Tween 80, preferably at a concentration of 0.01% (w/w).

As outlined, any of the above formulations can be further processed e.g. by lyophilization, spray drying or freezing, e.g. bulk freezing. The resulting processed product has characteristics derived from the liquid starting formulation, as defined above. Where necessary, additional agents may be included for the further processing, e.g. cryoprotectants, etc.

The formulations of the present invention preferably are suitable for use in methods of therapy of the animal or human body. Hence, the invention pertains to pharmaceutical or diagnostic compositions comprising a formulation of the polypeptide according to any aspect of the invention, or obtainable by any method or process of the invention.

The formulations of the invention are preferably pharmaceutical formulations. In particular embodiments, the formulations are suitable for parenteral administration to a human, e.g. subcutaneous, intravenous, intramuscular or intraperitoneal administration, preferably intravenous or subcutaneous administration. Administration encompasses any way of administering a liquid formulation, in particular injection.

To be suitable as a pharmaceutical formulation, the formulation of the invention will typically comprise the polypeptide of the invention (i.e. the active agent) in a suitable ratio to the volume. For example, for subcutaneous injection the concentration of active agent may be higher, in order to allow the necessary pharmaceutical dose to be administered in a smaller volume, as compared to a formulation for intravenous injection. However, in some embodiments the concentration of active agent will be identical for subcutaneous or intravenous injection, and can be in the exemplary ranges as defined above.

In some embodiments, the formulations of the invention may comprise additional agents, e.g. additional active agents, stabilizers, preservatives such as antimicrobial agents, etc.

The following table provides some non-limiting examples of citrate buffer based formulations of the present invention. All formulations can be adjusted to an osmolality of 290±60 mOsm/kg by adding a suitable excipient, if desired. The formulations can comprise any one or more of the polypeptides of the present invention, e.g. SEQ ID No. 2-5, particularly SEQ ID No. 2, or constructs comprising the same.

| | Buffer concentration | |
|---|---|---|
| Buffer | (mM) | pH |
| Citrate | 10 | 5.5 |
| Citrate | 10 | 6.0 |
| Citrate | 10 | 6.5 |
| Citrate | 20 | 5.5 |
| Citrate | 20 | 6.0 |
| Citrate | 20 | 6.5 |
| Citrate | 30 | 5.5 |
| Citrate | 30 | 6.0 |
| Citrate | 30 | 6.5 |
| Citrate | 40 | 5.5 |
| Citrate | 40 | 6.0 |
| Citrate | 40 | 6.5 |
| Citrate | 50 | 5.5 |
| Citrate | 50 | 6.0 |
| Citrate | 50 | 6.5 |

The buffer concentrations in this table are understood to optionally encompass ±5 mM. The pH values are understood to optionally encompass ±0.2. Each of the above buffers can be combined with one or more excipients selected from e.g. NaCl at a concentration of e.g. 50, 60, 70, 75 or 80 mM; mannitol at a concentration of e.g. 1, 2, 3, 4 or 5% (w/w); and sucrose at a concentration of e.g. 2, 3, 4, 5, or 6% (w/w), or a surfactant, e.g. a non-ionic surfactant, at a concentration of 0.001, 0.002, 0.003, 0.004, 0.005%, 0.01%, 0.02%, 0.05%, 0.08% or 0.1% (w/w), e.g. Tween-80.

The following table provides some non-limiting examples of phosphate buffer based formulations of the present invention. All formulations can be adjusted to an osmolality of 290±60 mOsm/kg by adding a suitable excipient, if desired. The formulations can comprise any one or more of the polypeptides of the present invention, e.g. SEQ ID No. 2-5, particularly SEQ ID No. 2, or constructs comprising the same.

| | Buffer concentration | |
|---|---|---|
| Buffer | (mM) | pH |
| phosphate | 10 | 6.5 |
| phosphate | 10 | 7.0 |
| phosphate | 10 | 7.5 |
| phosphate | 20 | 6.5 |
| phosphate | 20 | 7.0 |
| phosphate | 20 | 7.5 |
| phosphate | 30 | 6.5 |
| phosphate | 30 | 7.0 |
| phosphate | 30 | 7.5 |
| phosphate | 40 | 6.5 |
| phosphate | 40 | 7.0 |
| phosphate | 40 | 7.5 |
| phosphate | 50 | 6.5 |
| phosphate | 50 | 7.0 |
| phosphate | 50 | 7.5 |

The buffer concentrations in this table are understood to optionally encompass ±5 mM. The pH values are understood to optionally encompass ±0.2. Each of the above buffers can be combined with one or more excipients selected from e.g. NaCl at a concentration of e.g. 50, 60, 70, 80, 90 or 100 mM; mannitol at a concentration of e.g. 1, 2, 3, 4 or 5% (w/w); and sucrose at a concentration of e.g. 2, 3, 4, 5, 6, 7 or 8% (w/w) or a surfactant, e.g. a non-ionic surfactant, at a concentration of 0.001, 0.002, 0.003, 0.004, 0.005%, 0.01%, 0.02%, 0.05%, 0.08% or 0.1% (w/w), e.g. Tween-80.

### EFFECTS OF THE INVENTION

The invention provides stable formulations of the immunoglobulin single variable domains as defined herein, e.g. SEQ ID No. 2-5, in particular SEQ ID No. 2 or constructs comprising the same. “Stable” generally means that the immunoglobulin single variable domains do not suffer from physical or chemical changes upon storage for prolonged periods of time, e.g. 6 months to 36 months, such as 12, 24, 30 or 36 months. The formulations remain stable, even if exposed to one or more chemical or physical stresses such as elevated temperatures (equal to or higher than 25° C.). The formulations can also provide stability against physical stress such as shaking or stirring. In one embodiment “stable” means that the immunoglobulin single variable domains remains within the product characteristics with respect to purity and/or homogeneity as defined in Table 11.

More in particular “stable” means that upon storage for prolonged periods (as defined) under conditions (as defined) there is only a limited formation (as defined) of one or more of degradation products, e.g. low molecular weight derivatives (fragments) of the polypeptides of the invention; and/or chemical derivatives such as e.g. pyroglutamate derivatives; and/or high molecular weight derivatives (oligomers or polymers) formed e.g. by aggregation.

The skilled person is well acquainted with techniques to assess protein size, e.g. size exclusion chromatography-HPLC or to assess the formation of chemical derivatives, e.g. reversed phase HPLC. The skilled person is also familiar with commonly used apparatuses and software tools for performing such analyses. For example, the skilled person knows commonly used software to analyse chromatographic runs e.g. in terms of relative peak size. Examples include (but are not limited to) Agilent 1200 HPLC system equipped with ChemStation software (Agilent Technologies, Palo Alto, USA, Rev B) or Dionex Ultimate 3000 HPLC system equipped with Chromeleon software (Dionex Corporation, Sunnyvale, Calif., USA, V6.8).

General techniques that can be used to assess stability of an immunoglobulin single variable domain include static light scattering, tangential flow filtration, fourier transform infrared spectroscopy, circular dichroism, urea induced protein unfolding, intrinsic tryptophan fluorescence and/or 1-anilin-8-naphtalenesulfonic acid protein binding. In addition, the formulation of the invention shows little or no loss of potency/biological activity in the course of storage and/or under influence of one or more stresses as defined herein. Biological activity and/or potency can be determined e.g. as described in WO 09/138,519.

### Thermal Stability (Tm)

The formulations of the present invention are characterized by providing a high thermal stability of the immunoglobulin single variable domains as defined herein. Thermal stability can be evaluated e.g. by determining the melt temperature e.g. Tm. Suitable techniques for determining the melt temperature are known and include e.g. a thermal shift assay (TSA) e.g. as described herein. More specifically, the formulations of the present invention lead to an increase of Tm for the immunoglobulin single variable domains as determined by TSA in comparison to other formulations. This effect is exemplified in Table 2 of the experimental section.

As can be ascertained from the experimental section, high thermal stability, i.e. high Tm can be taken as an indication for storage stability.

According to the present invention, the formulations of the invention have a positive influence on Tm over a broad range of pH values, e.g. between 5.0 and 6.5 for citrate buffer, and 6.0 to 7.0 for phosphate buffer. The most advantageous effect on Tm can be observed for citrate buffer at pH 6.0±0.2 and phosphate buffer at pH 6.5 to 7.0, in particular 7.0±0.2.

The addition of excipients can have a further positive effect on Tm. For example, NaCl can increase Tm (in the context of a particular buffer) e.g. between 250 mM and 500 mM. At lower concentrations NaCl (e.g. as defined above or as exemplified in Table 2) has no negative effect on Tm and thus can be used in combination with the buffers in particular embodiments of the invention.

Mannitol or sucrose had a clear positive effect on Tm. These excipients can find use in particular embodiments of the invention, e.g. formulations where a bulking agent or lyoprotectants are advantageous. These exemplary embodiments do not preclude the use of further known lyoprotectants or bulking agents, either alone or in combination with mannitol or sucrose.

As can be ascertained from the experimental section, e.g. Table 3, increasing buffer strength was associated with an increase in Tm, e.g. in a range of between 40.0 mM and 66.7 mM, e.g. 50 mM. However, for a pharmaceutical application the skilled person will generally aim at a lower buffer strength, such as 5-50 mM. Thus, in preferred embodiments of the invention the formulations have a buffer strength in this range. In particular embodiments, the formulations have a buffer concentration that is as high as acceptable for pharmaceutical purpose, e.g. in a pharmaceutical formulation suitable for parenteral (e.g. intravenous or subcutaneous) injection in order to increase thermal stability.

As evidenced by the experimental section of this description, Tm as determined by TSA serves as a valuable indicator for stability of the immunoglobulin single variable domains of the invention. Increasing Tm indicates increased stability also in other physicochemical parameters, and can therefore indicate particularly preferable embodiments of the invention.

### Stability as Concerns Mechanical Stress

The formulations of the invention are characterized by a high stability as concerns mechanical stress, such as stirring, shaking or shear stress. One possible assay to evaluate stability under mechanical stress is the use of a fluorometer measuring scatter under 90° angle (500 nm). An increase in absorption reflects the formation of aggregates. When aggregates are formed, the increase over time can be determined. In certain embodiments the increase over time follows a linear curve for which a slope (absorbance units/s) can be determined. The formulations of the present invention are characterized by a slope of less than 0.006, e.g. less than 0.005, e.g. between 0 and 0.001. Another assay includes UV spectrophotometry e.g. at 280/340 nm.

At an exemplary, non limiting concentration of 10 mg/ml, the formulations of the invention only form reversible aggregates in response to stirring (e.g. at 4° C. 1 h stirring in 200 μl volume). After dilution, e.g. 1/10 dilution, or storage for 12 h at 4° C., no turbidity can be detected e.g. by UV spectrophotometry e.g. at A340 nm. Thus, the formulations of the invention prevent the formation of irreversible aggregates under mechanical stress.

The formulations comprising citrate buffers are particularly preferable and have a positive effect on protein recovery after e.g. stirring as defined above. For example, recovery is at least 90%, 95%, 98% or 100%. Protein recovery is determined in comparison to the total protein content before stressing the sample e.g. by stirring. The formulations comprising phosphate buffers result in a recovery of at least 75%, 80% or 85% after stirring as defined above.

In a further embodiment of the invention, the formulations of the invention may comprise a non-ionic detergent as defined above, e.g. Tween 80, e.g. at a concentration as defined above, e.g. 0.01% w/w. The addition of the detergent can further improve physical stability of the formulation. E.g. at a non-limiting exemplary concentration of 1 mg/ml, the addition of the detergent can prevent the formation of aggregates (reversible and irreversible) as determined e.g. by UV spectrophotometry (A340 nm). When stirring in volumes ranging from 1 ml to 10 ml, the addition of detergent is also expected to prevent the formation of aggregates.

As can be ascertained from Table 7 and the associated description, physical stability of the formulations of the present invention can also be demonstrated by RP-HPLC and SE-HPLC. Different non-limiting formulations of immunoglobulin single variable domains of the present invention (e.g. as defined in Table 4—all containing 0.01% w/w Tween-80) can withstand mechanical stress, e.g. stirring stress, without forming oligomers or degradation products. For example, these results can be obtained when the immunoglobulin single variable domains are subjected to vigorous stirring (e.g. stirring in a small glass vial at +4° C.; concentration=10 mg/mL; volume=200 μL). The formulations of the invention remain stable without degradation or oligomerization, as determined e.g. after 2 hours of stirring by RP-HPLC and SE-HPLC analysis. Exemplary data are shown in Table 7.

Although in some aspects of the invention, stirring may result in protein loss (potentially due to precipitation), no oligomerization or degradation (e.g. as determined by SE-HPLC or RP-HPLC profile respectively) is detected in any of the formulations. Overall, the best recovery can be obtained in D-PBS (90.3-94.9%) followed by citrate buffer (76.1-98.3%) and phosphate buffer (57.7-70.6%). Thus, according to a preferred embodiment of the invention, the formulations comprise a citrate buffer and show a recovery of at least 70%, 75%, 80%, 85%, 90%, 95%, 98%, e.g. 76.1-98.3%, e.g. under conditions as described above, wherein recovery is determined e.g. by RP-HPLC or SE-HPLC, as exemplified in Table 7. Some variation in recovery was seen between the different excipients. Advantageously, the excipient in the context of a citrate buffer can be mannitol, and recovery as defined above is at least 80%, 85%, 90%, 95% or 98%, e.g. 89.6-98.3%. In citrate buffer the presence of mannitol resulted in the highest recovery (89.6-98.3%) while in phosphate buffer this excipient gave the lowest recovery (57.7-63.1%).

### Storage Stability

The formulations of the invention provide for good stability when stored, e.g. at a temperature of −70° C., −20° C., 4° C., 25° C. or 40° C., e.g. for 1-24 months, preferably more than 24 months, such as e.g. 36 months or more, such as 1, 3, 6, 9, 12, 18, 24, 30, 36 months or more. More specifically, at temperatures −70° C., −20° C. and 4° C. storage stability is observed for 24 months, 30 months, 36 months or more. The most advantageous results can be obtained with citrate buffer based formulations, e.g. formulations 4-6 as exemplified in the experimental section. The skilled person can recognize that in the below discussion the preferred values reflect citrate buffer compositions, as exemplified in Table 8.

The skilled person will also recognize that storage at +25° C., and more in particular +40° C. represent stressed storage conditions. Such conditions are expected to increase and accelerate any signs of instability, e.g. chemical or physical instability. Hence, relatively short storage at e.g. +25 or +40° C. provides a good indication for storage stability under milder conditions (e.g. 4° C. or frozen).

Storage Stability in Terms of Protein Recovery For example, the formulations of the present invention provide for a protein recovery of at least 95%, e.g. at least 96, 97, 98, 99 or 100% after storage at a temperature between −70° C. and +40° C. Protein recovery can be determined by any known means to quantify proteins, e.g. by RP-HPLC or SE-HPLC, as exemplified in Table 8. These results can be observed e.g. after storage at the indicated temperature of 1 month, 7 weeks, 3 months, 6 months, 9 months, 12 months, 24 months, 30 months, 36 months or even more.

### Storage Stability in Terms of Chemical Derivatives/Degradation Products

Moreover, the formulations of the present invention provide for a production of chemical derivatives, e.g. pyroglutamate derivatives, of less than 2.5% in peak area as determined e.g. by RP-HPLC (as exemplified in Table 8). In this type of analysis, the area of a given peak is compared to the total area of the chromatogram, and a relative area % is allocated to each peak. The skilled person knows suitable analyzing means, e.g. suitable software, to analyze the chromatograms (specific, non-limiting examples include Agilent 1200 HPLC system equipped with ChemStation software (Agilent Technologies, Palo Alto, USA, Rev B) or Dionex Ultimate 3000 HPLC system equipped with Chromeleon software (Dionex Corporation, Sunnyvale, Calif., USA, V6.8). Thus, preferably, the pyroglutamate variant contributes to a peak area of less than 2%, preferably less than 1.5%, e.g. 1.2 or 1.3% as determined by RP-HPLC upon storage at temperatures between −70° C. and +5° C., e.g. +5° C., e.g. after storage for a duration as defined above, e.g. 7 weeks, 3 months or 6 months; or the pyroglutamate variant contributes to a peak area of 3% or less, such as less than 2.5%, preferably less than 2%, e.g. 1.8 or 1.7% as determined by RP-HPLC upon storage at temperatures between −70° C. and +25° C., e.g. +25° C., e.g. after storage for a duration as defined above, e.g. 7 weeks or 3 months, or 2.3% or 3.0% after storage for a duration of e.g. 6 months or 9 months; or the pyroglutamate variant contributes to a peak area of less than 12%, preferably less than 10%, more preferably less than 5% (e.g. in citrate buffer), e.g. 4.7 or 4.4% as determined by RP-HPLC upon storage at temperatures between −70° C. and +40° C., e.g. 40° C., e.g. after storage for a duration as defined above, e.g. 7 weeks, and less than e.g. 8%, e.g. less than 7.5% after storage at +40° C. up to a duration of 3 months, less than 12%, e.g. 11% after storage in citrate buffer at +40° C. for up to 6 months, and less than 16% after storage in citrate buffer at +40° C. for up to 9 months.

The formulations of the invention also provide for the absence of degradation products (as determined e.g. by RP-HPLC) over a storage period as defined above, e.g. 7 weeks, 3 months 6 months or 9 months at a temperature between −70° C. and +5° C., e.g. +5° C.; the formation of degradation products (as determined e.g. by RP-HPLC) of 2.5% or less, preferably less than 2%, e.g. less than 1.5%, e.g. 1.2 or 1.1% peak area upon storage at temperatures between −70° C. and +25° C., e.g. +25° C., e.g. after storage for a duration as defined above, e.g. 7 weeks, 3 months 6 months, or 9 months; or the formation of degradation products (as determined e.g. by RP-HPLC) of less than 10% peak area, preferably less than 8% in phosphate buffer, more preferably less than 4% (in citrate buffer), e.g. less than 3.5%, e.g. 3.2, 3.1 or 2.8% upon storage at temperatures between −70° C. and +40° C., e.g. +40° C., e.g. after storage for a duration as defined above, e.g. 7 weeks or less than 5%, e.g. less than 4.5% upon storage up to three months, less than 11%, e.g. 10% after storage in citrate buffer at +40° C. for up to 6 months, or less than 13%, e.g. 12.8% after storage in citrate buffer at +40° C. for up to 9 months.

The formulations of the invention also provide for storage stability, such that no degradation products (as defined e.g. by SE-HPLC) are formed at storage temperatures between −70° C. and +5° C., after storage durations as defined above, e.g. 7 weeks, 3 months, 6 months or 9 months; or less than 3% peak area, preferably less than 2.5%, e.g. 2.6, 2.7, 1.9 or 0%, most preferably 0% degradation products are formed (as defined e.g. by SE-HPLC) at storage temperatures between −70° C. and +25° C., e.g. +25° C., after storage durations as defined above, e.g. 7 weeks, 3 months, 6 months or 9 months; or less than 15%, such as less than 11% or less than 10%, preferably less than 5%, e.g. 4.0, 4.1 or 4.3% degradation products are formed (as defined e.g. by SE-HPLC) at storage temperatures between −70° C. and +40° C., e.g. +40° C., after storage durations as defined above, e.g. 7 weeks, 3 months, 6 months or 9 months.

### Storage Stability in Terms of Oligomerization

The formulations of the invention also provide for storage stability, such that no soluble oligomeric material is formed (as defined e.g. by SE-HPLC) at storage temperatures between −70° C. and +25° C., after storage durations as defined above, e.g. 7 weeks, 3 months, 6 months or 9 months; or less than 1% peak area, preferably less than 0.5%, e.g. 0.3% soluble oligomeric material is formed (as defined e.g. by SE-HPLC) at storage temperatures between −70° C. and +40° C., e.g. +40° C., after storage durations as defined above, e.g. 7 weeks, 3 months, 6 months or 9 months.

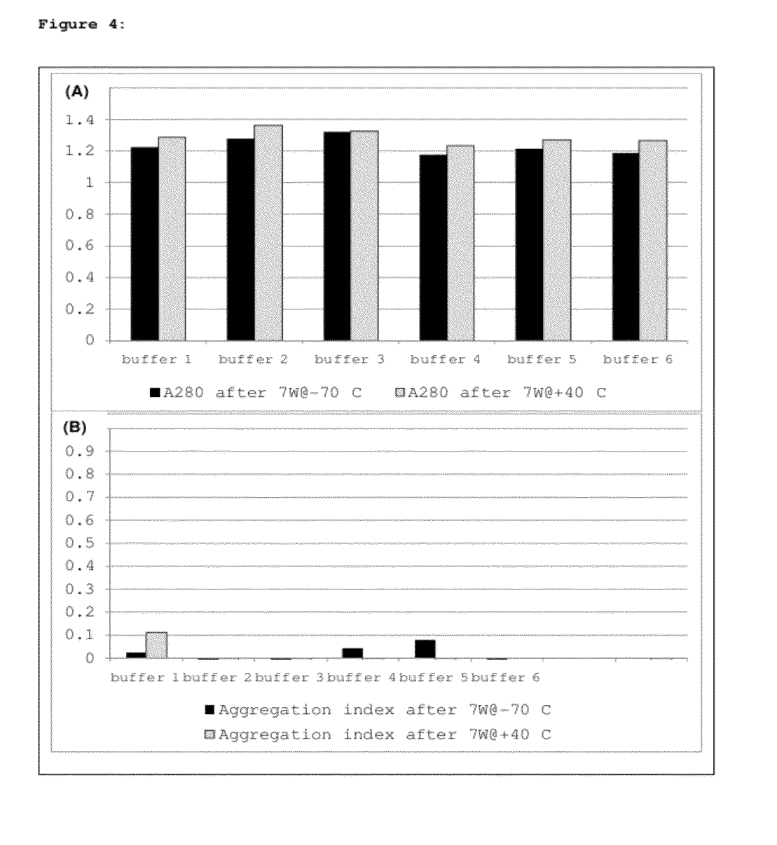

The present invention also has the effect of providing an aggregation index as determined by absorbance values [(100×A340)/(A280-A340)] which remains below 0.15, preferably below 0.1 after storage at −70° C. or +40° C. for storage of a duration as defined above, e.g. 7 weeks. Exemplary data can be seen in FIG. 4.

### Storage Stability as Reflected in Recovery of Main Product

Thus, the formulations of the invention have the effect that the main product peak, as determined e.g. by SE-HPLC (as exemplified in Table 8) is 100% of peak area after storage between −70° C. and +5° C. after a storage duration as indicated above, e.g. 7 weeks, 3 months, 6 months or 9 months; or the main product peak, as determined e.g. by SE-HPLC (as exemplified in Table 8) is at least 95% peak area, e.g. at least 97%, more preferably 100% after storage between −70° C. and +25° C., e.g. +25° C. after a storage duration as indicated above, e.g. 7 weeks, 3 months, 6 months or 9 months; or the main product peak, as determined e.g. by SE-HPLC (as exemplified in Table 8) is at least 85% of peak area, at least 90%, preferably at least 95%, after storage between −70° C. and +40° C., e.g. +40° C., after a storage duration as indicated above, e.g. 7 weeks, 3 months, 6 months or 9 months.

The formulation according to the present invention also has the effect that the main peak as determined by RP-HPLC after storage e.g. at a concentration of 10 mg/ml at between −70° C. and +25° C. for between 1 and nine months remains unchanged as compared to the formulation prior to storage, and represents at least 90% of peak area, more preferably at least 95% of peak area, wherein the reference sample has a main peak of e.g. 95% peak area. Upon storage at +40° C. for 1 month the formulation of the present invention retains the main peak as determined by RP-HPLC of at least 80% peak area, 85% or 90%; after storage for 2 months of at least 80%, or 85%, and after storage for 3 months of at least 75% or 80%.

Moreover, as determined by cIEF, the formulation of the present invention has the effect of providing recovery of the main product after storage at a concentration of e.g. 10 mg/ml for between 1-9 months at a temperature between −70° C. and +25° C. that is comparable to the reference sample (formulation without storage, main peak is at least 98% of peak area), e.g. a main peak of at least 90% peak area, preferably at least 95%. After storage for 1 month at +40° C., the main peak is at least 85%, or at least 88%, after storage at +40° C. the main peak is at least 70% and after storage for 3 months of at least 65% or 70%.

### Storage Stability Under Freeze-Thaw Conditions

Apart from providing stability of the formulations under conditions of storage that remain constant over time (e.g. storage at 4° C.), or include a single freeze thaw cycle (e.g. storage at −20 or −70° C.), a further effect of the invention is stability under conditions of repeated freeze thaw cycles. Every transition between frozen and liquid state and vice versa imposes particularly stressful conditions upon the immunoglobulin single variable domains.

The formulations of the invention also have the effect of providing good stability under freeze/thaw conditions. For example the formulations of the invention can be subjected to e.g. 10 freeze/thaw cycles between −70° C. and room temperature (e.g. 25° C.), or −20° C. and room temperature. The immunoglobulin single variable domains comprised in the formulations will withstand these conditions without significant deterioration, as ascertained e.g. by RP-HPLC or SE-HPLC. Exemplary data for 6 different non-limiting embodiments of formulations of the invention are shown in Tables 5 and 6, and reveal that in all cases chemical and physical integrity of the immunoglobulin single variable domains has been preserved. Overall recovery was in the range between 95 and 100% determined by comparing the total surface peak area on RP-HPLC or SE-HPLC with that of a reference sample, preferably at least 95, 98 or 99%. The relative proportion of the different peaks as exemplified in Tables 5 and 6 remained unchanged in comparison to a control subjected to only one freeze/thaw cycle.

More specifically, at a concentration of between 10 mg/ml and 0.5 mg/ml, 5 or 10 freeze thaw cycles will result in a recovery (as determined on the basis of e.g. total area) of polypeptide, as determined either by RP-HPLC or SE-HPLC that is at least 90%, 95%, 98% or 100%; wherein in a particular embodiment the RP-HPLC or SE-HPLC profile was unchanged as compared to a reference sample (1 freeze thaw cycle).

### Stability in Terms of Potency

The skilled person knows various ways to determine potency of target specific polypeptides. Common assays relate to target binding, or measure biological effects associated with target binding. In the case of polypeptides binding to CXCR4, in particular immunoglobulin single variable domains, more specifically polypeptides according to any one of SEQ ID No. 2 to 5, e.g. SEQ ID No. 2 suitable assays are described for example in the experimental section of WO 09/138,519, e.g. examples 3, 4 and 5, experimental section of PCT/EP2011/050156, or experimental section of PCT/EP2011/050157). In one particular embodiment, potency of immunoglobulin single variable domains can be ascertained as follows:

In one embodiment, potency of the polypeptide of the present invention can be determined by binding to its antigen by a conventional assay, e.g. ELISA, Biacore, RIA, FACS, etc.

In one exemplary embodiment, cells expressing CXCR4, e.g. CHO-K1 cells, can be exposed to the polypeptides in the presence of a cytokine, e.g. SDF1-alpha, e.g. after serum starvation, e.g. for 18 hours. After incubation for a suitable period of time, e.g. 10 minutes, the cells can be lysed to assess the phospho-ERK1/2 pathway by conventional means. The effect on the phosphor-ERK1/2 pathway then serves as a readout for assessing potency.

The formulations of the present invention have the effect that the polypeptides as defined herein, e.g. according to any one of SEQ ID No. 2 to 5, e.g. SEQ ID No. 2, can be subjected to at least 10 freeze-thaw cycles without significant loss of potency. More specifically, at a concentration of e.g. 10 mg/ml, 10 freeze thaw cycles between −70° C. and room temperature (e.g. 25° C.) result in a potency of at least 75%, 80%, 85% or 90%, e.g. at least 89.4% as compared to a reference sample (100%); or, alternatively, 10 freeze thaw cycles between −20° C. and room temperature result in a potency of at least 80%, 85%, 90%, 95% or 98% e.g. at least 98,7% as compared to a reference sample. At a concentration of e.g. 0.5 mg/ml, 10 freeze thaw cycles between −70° C. and room temperature result in a potency of at least 85%, 90%, 95% or 100%, as compared to a reference sample; or, alternatively, 10 freeze thaw cycles between −20° C. and room temperature result in a potency of at least 85%, 90%, 95% or 100% as compared to a reference sample. In all cases the reference sample underwent only a single freeze-thaw cycle as defined above.

The formulations of the present invention can be stored for 1-6 months, e.g. 3 months, 6 months or 9 months at a temperature of 40° C. and retain a potency of at least 70%, 75%, 85%, 90%, 95% or 100% as compared to a reference sample stored e.g. at −70° C. For example, a formulation of the present invention can be stored at +40° C. for 6 months or 9 months, and will show remaining potency of e.g. at least 70% and at least 60%, respectively. Under these conditions, the formulations may exhibit degradation of up to 10% peak area, and up to 11% peak area pyroglutamate formation e.g. after 6 months of storage.

At −70° C., −20° C., +5° C. and +25° C. the formulations of the present invention will exhibit a potency of at least 85%, 90%, 95% or 100% for up to 6 months or even 9 months. Degradation and pyroglutamate formation remains within the values described for three month storage at the respective temperatures, as stated above.

### Stability of Half-Life Extended Embodiments

In certain embodiments the present invention relates to polypeptide constructs comprising one or more immunoglobulin single variable domains, directed against one or more target antigens comprising, but not limited, to CXCR4, and a further polypeptide sequence capable of extending half-life by binding to a half life extending target, such as serum albumin. In these constructs, the “serum-albumin binding peptide or binding domain” may be any suitable serum-albumin binding peptide or binding domain capable of increasing the half-life of the construct (compared to the same construct without the serum-albumin binding peptide or binding domain), and may in particular be serum albumin binding peptides as described in WO 2008/068280 by applicant (and in particular WO 2009/127691 and the non-prepublished U.S. application 61/301,819, both by applicant), or a serum-albumin binding immunoglobulin single variable domain (such as a serum-albumin binding Nanobody; for example Alb-1 or a humanized version of Alb-1 such as Alb-8, for which reference is for example made to WO 06/122787). Further specific examples of serum albumin binding immunoglobulin single variable domains are specified above.

The formulations of the present invention have the particular advantage in the context of the peptides as described in WO 2008/068280 by applicant (and in particular WO 2009/127691 and the non-prepublished U.S. application 61/301,819, both by applicant) or a serum-albumin binding immunoglobulin single variable domain (such as a serum-albumin binding Nanobody; for example Alb-1 or a humanized version of Alb-1 such as Alb-8 (including tag-less versions thereof) and the further half life extending binders as listed herein, for which reference is for example made to WO 06/122787) that they prevent or reduce oligomerization, in particular dimerization. More in particular, the formulations based on citrate buffer as described herein serve to have this beneficial effect.

### Stability in Terms of Compatibility

The formulations of the present invention also have the effect of good compatibility with a range of different diluents including physiological saline or pharmaceutically acceptable buffers. E.g. the formulations can be mixed/diluted with such diluents, without affecting chemical and physical stability of the immunoglobulin single variable domains. The respective exemplary data can be observed in Table 9.

Thus, the formulations of the present invention also provide stability over a broad range of concentrations, as defined herein.

### Summary of Stabilizing Effects

The formulations of the present invention have the effect of maintaining the polypeptides of the present invention within product characteristics with respect of purity and/or homogeneity even after prolonged storage, e.g. for durations as defined above, at temperatures between −70° C. and +25° C., wherein these product characteristics are as defined below:

| Analysis | Characteristic |
|---|---|
| RP-HPLC | ≧80.0% main peak, e.g. ≧90.0% main peak, preferably |
| | ≧95.0% main peak |
| SE-HPLC | ≧90.0% main peak; e.g. ≧95.0% main peak |
| cIEF | ≧80.0% main peak; e.g. ≧90.0% main peak |
| potency | 50-150% relative to reference standard; e.g. 60-120% |
| | or 70-100%, preferably 80-100% |

Storage of immunoglobulin single variable domains as defined herein, in particular 4CXCR104 at −70° C. for 7 weeks, 3 months, 6 months or 9 months did not affect their physicochemical characteristics for any of the formulations of the invention, in particular the six non-limiting examples of buffers tested in the experimental section. Storage did not have a significant effect on RP-HPLC, SE-HPLC or cIEF profiles.

Storage of immunoglobulin single variable domains as defined herein, in particular 4CXCR104 at +5° C. for 7 weeks, 3 months, 6 months or 9 months did not affect their physicochemical characteristics for any of the formulations of the invention, in particular the six non-limiting examples of buffers tested in the experimental section. Storage did not have a significant effect on RP-HPLC or SE-HPLC profiles.

Storage of immunoglobulin single variable domains as defined herein, in particular 4CXCR104 at +25° C. for 7 weeks, 3 months, 6 months or 9 months did not have an effect on protein recovery, although storage resulted in a slight increase in the amount of pyroglutamate variant and minor protein degradation. This storage effect was more pronounced in phosphate buffers (e.g. buffer 1-3) than in citrate buffers (e.g. buffer 4-6). SE-HPLC analysis did not detect any oligomers. No significant difference between excipients could be observed.

The results obtained for particular, non-limiting examples of analysis methods can be summarized as follows:

### RP-HPLC

RP-HPLC results of immunoglobulin single variable domains as defined herein, in particular 4CXCR104 stored at +40° C. for up to 7 weeks, 3 months, 6 months or 9 months in any of the formulations of the invention, in particular the six non-limiting examples of buffers tested in the experimental section:
Storage under stressed conditions (+40° C.) did not appear to have a significant effect on protein recovery. The obtained surface areas were comparable with that of the reference.
Storage under stressed conditions (+40° C.) caused a gradual increase in degradation products and in the amount of pyroglutamate variant as well as other variants (increasing surface area of pre and post peaks.
Corresponding with the results obtained at +25° C., the effect of storage under stressed conditions (+40° C.) on the RP-HPLC profile of immunoglobulin single variable domains as defined herein, in particular 4CXCR104 was more pronounced in phosphate buffers (buffer 1-3) than in citrate buffers (buffer 4-6). FIGS. 5 (A) and (B) shows a graphic representation of the kinetics of pyroglutamate formation and degradation in the 6 buffers.
Again, no significant difference between excipients could be observed.

### SE-HPLC

Immunoglobulin single variable domains as defined herein, in particular 4CXCR104 stored at +40° C. for up to 7 weeks in any of the formulations of the invention, in particular the six non-limiting examples of buffers tested in the experimental section, was analyzed by SE-HPLC:
Storage under stressed conditions (+40° C.) did not appear to have a significant effect on protein recovery: the surface areas for the stressed samples were comparable with that of reference (see the preceding table).
Storage under stressed conditions (+40° C.) caused a gradual increase in degradation products (increasing surface area of post peaks) which was more pronounced in phosphate buffers (buffer 1-3) than in citrate buffers (buffer 4-6) (confirming the results obtained at +25° C., and the results obtained by RP-HPLC).
A small population of oligomers was detected in buffer 2 and buffer 3 (containing sucrose) indicating that a formulation of phosphate with mannitol or phosphate with sucrose is more prone to oligomerization.
   cIEF

Immunoglobulin single variable domains as defined herein, in particular 4CXCR104 stored at +40° C. for up to 1 month in any of the formulations of the invention, in particular the six non-limiting examples of buffers tested in the experimental section was analyzed by cIEF electropherograms:
Stressed storage at +40° C. did not appear to have a significant effect on protein recovery (resulting surface areas comparable with that of reference).
Stressed storage at +40° C. caused an increase in pre and post peaks which was more pronounced in phosphate buffers (buffer 1-3) than in citrate buffers (buffer 4-6).
The highest purity was observed in the citrate buffers, confirming SE-HPLC and RP-HPLC data.

### Methods of the Invention

The amino acid sequences of the invention can be produced by any commonly used method. Typical examples include the recombinant expression in suitable host systems, e.g. bacteria or yeast. The amino acid sequences will undergo a suitable purification regimen prior to being formulated in accordance to the present invention.

The present invention encompasses methods of producing the formulations as defined herein.

The purification and formulation steps may coincide, e.g. when the amino acid sequences of the invention are eluted from a column using a buffer according to the present invention. Alternatively, the formulations of the invention can be prepared by exchanging a buffer by any suitable means, e.g. means widely used in the art such as dialyzing, ultrafiltration, etc.

In some embodiments the method of producing a formulation of the invention may also relate to the reconstitution of a lyophilized or spray dried formulation, e.g. by addition of water or a suitable buffer (which may optionally comprise further excipients).

The method for preparing a formulation according to the present invention may encompass further steps, such as filling it into vials suitable for clinical use, such as sealed containers and/or confectioning it in a dosage unit form. The method may also comprise further steps such as spray drying, lyophilization, or freezing, e.g. bulk freezing. The invention also encompasses the containers, dosage unit forms, or other products obtainable by any of the methods recited herein.

The formulations of the present invention can be used to store the polypeptides as defined herein. Thus, the invention encompasses a method of storage of a polypeptide as used herein, characterized by the use of a formulation as defined herein. More specifically, the invention encompasses methods for stabilizing a polypeptide as defined herein for storage, comprising e.g. the preparation of a formulation as described herein. Storage can be 1-24 months, more than 24 months or even more than 36 months, such as 1, 3, 6, 9, 12, 24, 30, 36 months or more, e.g. at least 3 months or 6 months, optionally at a temperature between −70° C. and +40° C., such as −70° C., −20° C., +5° C., +25° C. or +40° C., preferably a temperature between −70° C. and +25° C., more preferably at a temperature between −20° C. and 4° C. Thus, storage may encompass freezing, freeze-drying (lyophilization) and/or spray drying. The storage methods may furthermore comprise the assessment of physical and chemical integrity of the polypeptides as defined herein.

The present invention also relates to methods for analyzing formulations comprising at least one of the polypeptides as defined herein. The formulations can be analyzed for any signs of chemical or physical instability of the polypeptides, as defined herein. For example, the formulations can be assessed for the presence of degradation products, e.g. low molecular weight derivatives such as proteolytic fragments; and/or for chemical derivatives, e.g. pyroglutamate derivatives; and/or for high molecular weight derivatives such as aggregates, agglomerates, etc. The formulation can also be assessed for total protein content and/or potency. Each of the various assay methods as referred to herein can be used in the analysis method of the present invention.

Thus, the present invention also relates to a method for monitoring and/or assessing the quality and/or stability of a formulation, e.g. during one or more of manufacture, storage and use. The invention also relates to a method of quality control of a formulation, e.g. to assess that the formulation remains within product characteristics as further described herein. The invention in any of these aspects comprises one or more selected from the comparison with one or more reference samples, the analysis of batch to batch variation, and the ongoing monitoring of a production process.

### Medical Uses/Pharmaceutical Compositions

In certain embodiments the present invention includes the use of the formulations of the present invention in therapy, i.e. in methods of treating a human or animal subject. The invention also relates to pharmaceutical compositions comprising the formulations as described herein, which may be liquid solutions of the polypeptides as defined herein. Liquid solutions will in particular be suitable for parenteral administration, e.g. injection and/or infusion. Other forms of systemic administration, e.g. via implantable devices, micro-infusion pumps (optionally implantable), and/or (implantable) sustained release formulations, e.g. deposits, gels, biodegradable polymer formulations are also within the scope of the present invention. Pharmaceutical compositions are sterile and stable during manufacture and storage, as derivatives/degradation products of the polypeptides are undesired in a clinical setting. The composition will also be of high purity, e.g. exclude the presence of bacterial products such as LPS. The formulations can be sterilized by any suitable means, e.g. sterile filtration, irradiation, combinations thereof, etc.

For the role of CXCR-4 and anti-CXCR-4 therapy in various forms of cancer, further reference is for example made to the reviews by Burger and Kipps, Blood, 2006; Dorsam and Gutkind 2007, Nat Rev Cancer, 2007; Kryczek et al, Am J Physiol Cell Physiol, 2007; Balkwill, Nat Rev Cancer, 2004; and for example to Mueller et al, Nature, 2001, 50-56 (breast cancer); Nervi et al., Blood, 2009, 119, 24, p. 6206-62-14 (AML); Redjal et al., Clin. Cancer Res., 2006, 12(22), 2006, 6765-6771 (gliomas); Rubin et al., PNAS, 100, 23, 20-03, 13513-13518 (brain tumors); Jin et al, Mol. Cancer. Ther. 2008; 7: 48-58 (CML) and Zeng et al., Blood, 113, 24, 2009, 6215-6224.

In a broader sense, the formulations can be used as a medicament to inhibit signaling that is mediated by human CXCR4 and/or its ligand(s); and/or in the prevention or treatment of diseases associated with an increased signalling of CXCR4, such as the various diseases in the group of cancer such as hematopoietic cancers like CLL, AML, ALL, MM, Non-Hodgkin lymphoma, solid tumors such as breast cancer, lung cancer, brain tumors, ovarian cancer, stromal chemoresistance of tumors, leukemia and other cancers, disrupting adhesive stromal interactions that confer tumor cell survival and drug resistance, mobilizing tumor cells form tissue sites and making them better accessible to conventional therapy, inhibiting of migration and dissemination of tumor cells (metastasis), inhibiting or paracrine growth and survival signals, inhibiting pro-angiogenesis effects of SDF-1, inflammation and inflammatory disorders such as bowel diseases (colitis, Crohn's disease, IBD), infectious diseases, psoriasis, autoimmune diseases (such as MS), sarcoidosis, transplant rejection, cystic fibrosis, asthma, chronic obstructive pulmonary disease, rheumatoid arthritis, viral infection, HIV, West Nile Virus encephalitis, common variable immunodeficiency. Furthermore, the amino acid sequences of the invention can be used for stem cell mobilization in various patients in need of stem cells after X-ray radiation such as e.g. cancer patients after radiation treatment to replenish the stem cell pool after radiation in cancer patients, or in patients in need of more stem cells, e.g. in patients with ischemic diseases such as myocardial infarction (MI), stroke and/or diabetes (i.e. patients in need of tissue repair) wherein more stem cell would be re-transfused (after mobilization, screening, selection for lineage in need (e.g. cardiac, vascular lineages) and ex-vivo expansion of patient's own stem cells).

For example, formulations as defined herein can be used for the treatment of cancer or AIDS.

Methods of treatment include the continuous (e.g. infusions, sustained release formulations) or intermittent administration (e.g. daily, thrice a week, twice a weekly, weekly, biweekly or once a month). In the case of polypeptides that are half-life extended even intermittent administration may result in continuous exposure to the polypeptide as defined herein, depending on the half life (e.g. T1/2β) in relation to the administration frequency. Variations in drug levels are still considered continuous administration, if the drug levels do not reach undetectable levels between administrations and, more specifically, remain at a therapeutically active concentration over the course of treatment (excluding the initial and terminal phases).

The formulations/pharmaceutical preparations/dosage unit forms and any other clinically relevant embodiments of the present invention may also comprise further active ingredients, e.g. drugs that are known in the treatment of cancer and/or AIDS.

### Further Products of the Invention

The present invention relates to any product that is associated with the formulations of the present invention, e.g. by comprising them, or by being necessary for their production or confectioning, without any limitations.

For example, the present invention relates to an article of manufacture, e.g. a sealed container comprising one or more of the formulations according to the present invention. The invention also relates to a pharmaceutical unit dosage form, e.g. a dosage form suitable for parenteral administration to a patient, preferably a human patient, comprising one or more of the formulation according to any embodiment described herein. The dosage unit form can be e.g. in the format of a prefilled syringe, or a vial. The syringe or vial can be manufactured of any suitable material, including glass or plastic. The invention also relates to a kit comprising one or more of the formulations according to the present invention. The kit may further comprise instructions for use and/or a clinical package leaflet. In any embodiment of the products as defined herein, the invention also encompasses the presence of packaging material, instructions for use, and/or clinical package leaflets, e.g. as required by regulatory aspects.

### ABBREVIATIONS

AA Amino Acid
° C. degrees Celsius
cIEF Capillary IsoElectric Focusing
DP Drug Product
D-PBS Dulbecco's Phosphate buffered saline (e.g. Gibco—Cat. No. 20012-043)
DSP DownStream Processing
εth 280 nm Theoret. Ext. coefficient at 280 nm (cm-1*(mg/mL)−1)
εexp 280 nm Experimental Ext. coefficient at 280 nm (cm-1*(mg/mL)−1)
FB Formulation Buffer
FT Freeze-Thaw
20GS 20 amino acid Glycine Serine linker joining Nanobody® building blocks
HPLC High Pressure Liquid Chromatography
IPC In-Process Control
IPM In-Process Monitoring
IV IntraVeneously
LC-MS Liquid Chromatography combined with Mass Spectrometry
mAU milli Absorption Units
MW Molecular Weight
pI Isoelectric Point
pIth Theoretically predicted Isoelectric Point
pIexp Experimentally determined Isoelectric Point
Q-PCR Quantitative Polymerase Chain Reaction
RP-HPLC Reversed Phase High Pressure Liquid Chromatography
RT Room Temperature
SE-HPLC Size-Exclusion High Pressure Liquid Chromatography
Tm Melting Temperature
TSA Thermal Shift Assay
USP UpStream Processing

The general principles of the present invention as set forth above will now be exemplified by reference to specific experiments and examples. However, the invention is not to be understood as being limited thereto.

The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Experimental Series 1

This experimental series summarizes the advantageous effects of particular formulations of 4CXCR104 Nanobody® (4CXCR1044) on overall stability at an exemplary protein concentration of 10 mg/mL, in particular a preferred formulation buffer for the 4CXCR104 drug product (DP) which is suitable for subcutaneous (SC) and intravenous (IV) administration, preferably IV bolus injection. The following characteristics of the 4CXCR104 Nanobody® were evaluated:
advantageous effects of formulations of the invention on thermal stability (Tm) containing different excipients by means of a fluorescence-based thermal shift assay (TSA)
freeze-thaw stability in formulations of the invention, i.e. up to 10 consecutive freeze-thaw cycles at −70° C. and −20° C.
stirring stability in formulations of the invention at +5° C. and at room temperature (with/without Tween-80)
storage stability in formulations of the invention at −70° C., −20° C., +5° C., +25° C. and +40° C.

Analysis of the different samples included:
appearance
pH
osmolality
content (A280/A340)
reversed-phase chromatography (RP-HPLC)
size exclusion chromatography (SE-HPLC)
capillary isoelectric focusing (cIEF)
liquid chromatography combined with mass spectrometry (LC-MS)

### Summary of Results

Phosphate (pH 6.0-7.0) and citrate (pH 5.5-6.5) buffer with relatively high buffer strength provide for the highest Tm for 4CXCR104, i.e. the highest thermal stability. The addition of NaCl, sucrose or mannitol had a positive effect on Tm.

A stirring experiment showed that the presence of 0.01% Tween-80 prevented sample turbidity during stirring at low concentration (1 mg/mL) demonstrating protection against mechanical, e.g. shear stress.

A first storage study was performed in phosphate and citrate buffer (including D-PBS as a reference) and showed that 4CXCR104 was less prone to degradation and pyroglutamate formation in citrate based formulations.

Furthermore, 4CXCR104 formulated in phosphate buffer with mannitol or sucrose demonstrated minor oligomerization after 2 months storage at +40° C. which could not be detected in any of the citrate buffers.

No significant effect of excipient type (NaCl, mannitol and sucrose) on freeze/thaw/storage/stirring stability was observed. Good freeze/thaw stability of the 4CXCR104 Nanobody® was observed for all the tested buffers.

In a particularly preferred embodiment the final formulation buffer of 4CXCR104 at 10 mg/mL is defined as 50 mM citrate pH 6.0 (1.325 g/L citric acid monohydrate+12.850 g/L tri-sodium citrate dihydrate), 75 mM NaCl (4.383 g/L) and 0.01% Tween-80 (w:w).

### 4CXCR104

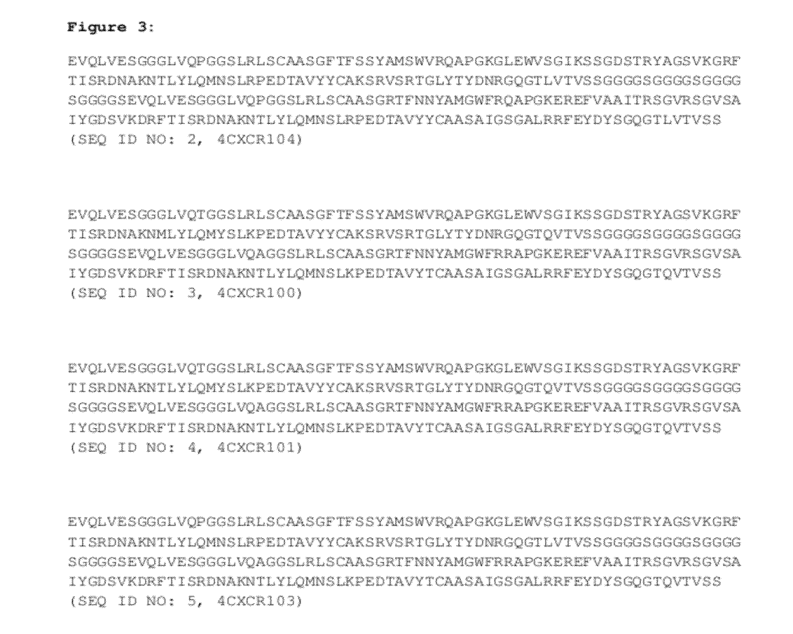

4CXCR104 (see also U.S. provisional application 61/358,495 with the filing date Jun. 25, 2010, in particular SEQ ID No. 7) is a biparatopic Nanobody® consisting of two fully sequence optimized variable domains of an anti-CXCR4 heavy-chain llama antibody, i.e. 4CXCR016 (D2 building block) and 4CXCR026 (D4 building block) (see FIG. 2). The subunits are fused by a 20GS linker. The sequence is shown in FIG. 3 (SEQ ID No. 2). The characteristics of the Nanobody® are as follows: 270 amino acids, MW=28111.07 Da, pIth=9.66, pIexp=10.3, εth 280 nm(0.1%)=1.24 cm-1 (mg/mL)−1, ε_{exp 280 nm} (0.1%)=1.41 cm⁻¹ (mg/mL)⁻¹.

### Analyte and Reagents

Test Items
Formulation: D-PBS (pH 7.2; 2.71 mM Na2HPO4-7H2O; 1.54 mM KH2PO4; 155.17 mM NaCl)
Concentration: 10.34 mg/mL
Formulation: 50 mM phosphate (pH 7.0)
Concentration: 21.3 mg/mL
Formulation: 50 mM citrate (pH 6.0)
Concentration: 21.4 mg/mL
Formulation: 50 mM citrate (pH 6.0)+75 mM NaCl+0.01% Tween-80
Concentration: 10.6 mg/mL

Reference Items

Unstressed samples of 4CXCR104 in D-PBS or other formulation buffer (stored at −70° C.) were used as reference for analysis of stability samples (freeze-thaw, stirring and storage).

### Methods

This section gives an overview of the analytical methods that were used during stability testing of the 4CXCR104 molecule (Table 1).

TABLE 1
Overview of the analytical methods for 4CXCR104.

| analytical | |
|---|---|
| method | Purpose |
| RP-HPLC | purity (variants) + |
| | content |
| SE-HPLC | Purity |
| | (aggregates/degradation) |
| cIEF | identity + purity |
| | (charge variants) |
| content | Concentration |
| (A280) | |
| potency | identity + biological |
| assay | activity |

### Thermal Shift Assay (TSA)

A TSA can be performed in a Roche LightCycler480 Q-PCR device to evaluate the effect of buffer (couple), ionic strength, pH and excipients on the thermal stability of proteins. The assay provides a Tm value (° C.) that is indicative for the thermal stability in the tested buffers. In short, the assay follows the signal changes of a fluorescence dye (for example in this case Sypro Orange) while the protein undergoes thermal unfolding. When Sypro Orange is added to a properly folded protein solution, it is exposed in an aqueous environment and its fluorescence signal is quenched. When the temperature rises, the protein undergoes thermal unfolding and exposes its hydrophobic core region. Sypro Orange then binds to the hydrophobic regions and unquenches, which results in an increase of a fluorescence signal.

The assay was performed on solutions containing the protein sample at 0.25-0.33 mg/mL and 33x-40x Sypro Orange. The denaturation program consisted of the following steps:
heating to +37° C. at a ramp rate of +4.4° C./s and hold for 10 s
heating to +90° C. at a continuous ramp rate of +0.02° C. (20 acquisitions per ° C.)
cooling to +37° C. at a ramp rate of −2.2° C./s and hold for 10 s

First derivative of the resulting fluorescence curves was calculated and melting temperatures were determined.

### Chromatographic Methods

The chromatographic methods employed herein are well known to the skilled person. For SE-HPLC, e.g. a TSK-Gel G2000SWXL (TOSOH Bioscience, 08540) column can be used. For RP-HPLC, e.g. a Zorbax 300SB-C8 (Agilent, 883995-906) column can be used. Standard HPLC equipment is commercially available. HPLC software used for data collection and integration of chromatograms can be exemplified (non limiting) by Agilent 1200 HPLC system equipped with ChemStation software (Agilent Technologies, Palo Alto, USA, Rev B); or Dionex Ultimate 3000 HPLC system equipped with Chromeleon software (Dionex Corporation, Sunnyvale, Calif., USA, V6.8).

### Potency Assay

The skilled person knows several different ways of determining the potency of CXCR4 specific immunoglobulin single variable domains (see for example experimental section of WO 09/138,519, e.g. examples 3, 4 and 5, or experimental section of PCT/EP2011/050157). In one particular embodiment, potency of immunoglobulin single variable domains can be ascertained as follows:

The potency of ALX-0651 is determined by the CHO-K1 erk potency assay, using the Cellul'Erk kit from Cisbio containing pre-made lysis buffer, blocking buffer, detection buffer and detection antibodies.

CHO-K1 cells (Discoverx) stably expressing CXCR4 are seeded at 10,000 cells/well in a 96-well white assay plate in complete medium (F12 medium, glutamine, streptomycin, 10% fetal bovine serum). Subsequently, cells are incubated for 24 hours at 37° C. and 5% CO2. The cells are serum starved in 50 μL medium for 18 hours following the 24 hour incubation step. Individual dilution series of the reference sample, control sample and test samples are prepared in a predilution plate, together with 10 nM SDF1-alpha (R&D Systems). These dilutions are transferred to the cells (50 μL/well) and the reaction mixture is incubated. After the incubation period of 10 minutes, the reaction mixture is removed by aspiration and 60 μL/well lysis buffer is added to the wells for 45 minutes. When lysis is completed, anti-phospho-ERK1/2 antibody labeled with a fluorescent dye d2 (1/100) and an anti-ERK1/2 antibody labeled with a fluorescent dye Eu3+-cryptate (1/200) are added (15 μL/well). After incubation of 2 hours, the read-out is performed using a time-resolved fluorescence measurement at 665 nm.

### Storage Conditions

The samples in the freeze/thaw stability and storage stability studies were kept in freezers (−70° C.±10° C. and −20° C.±5° C.), a cold room (5° C.±3° C.) and stability chambers (25° C.±2° C. and 37° C.±2° C.). At the time of analysis, the samples were transferred to a cold room (5° C.±3° C.) and analyzed as soon as possible (within 1 day).

### Results

### Advantageous Effects of Buffers and Excipients of the Invention

Advantageous effects of formulations of the invention on thermal stability of 4CXCR104 were shown in a number of TSA experiments in several buffers and in the presence of different excipients.

The following set of buffers and pH ranges were compared:

### Buffers of the Invention

citrate pH 5.5-6.0-6.5
phosphate pH 6.0-6.5-7.0

### Comparative Buffers

acetate pH 5.5
succinate pH 5.5-6.0-6.5
histidine pH 6.0-6.5

In addition, the following excipients were included in the comparison:
NaCl 75-150-250-500 mM
mannitol 2.5%
sucrose 5.0%

An overview of the obtained melting temperatures (Tm) is given in Table 2 and Table 3.

TABLE 2
Melting temperatures (° C.) of 4CXCR104 formulated in different buffer/excipient
combinations as determined by TSA.

| buffer | | no | 75 mM | 150 mM | 250 mM | 500 mM | 2.5% | 5% |
|---|---|---|---|---|---|---|---|---|
| (50 mM) | pH | excipients | NaCl | NaCl | NaCl | NaCl | mannitol | sucrose |
| Citrate | 5.0 | 61.31* | — | 61.13 | 61.13 | 61.55 | — | 62.33 |
| | 5.5 | 61.91 | 61.91 | — | — | — | 62.75 | 62.75 |
| | 6.0 | 62.35** | 62.33 | 61.96 | 62.38 | 63.21 | 62.75 | 63.16* |
| | 6.5 | 61.91 | 61.91 | — | — | — | 62.33 | 63.16 |
| phosphate | 6.0 | 61.91 | 61.91 | — | — | — | 62.75 | 62.75 |
| | 6.5 | 62.35** | 61.91 | 61.96 | 61.96 | 62.79 | 63.16 | 63.16* |
| | 7.0 | 62.48** | 61.91 | 61.96 | 61.96 | 62.79 | 63.16 | 63.37* |
| Acetate | 5.5 | 60.69* | — | 61.13 | 61.13 | 61.96 | — | 61.08 |
| succinate | 6.0 | 61.73* | — | 61.96 | 61.96 | 62.79 | — | 62.33 |
| histidine | 6.0 | 59.86* | — | 60.72 | 61.13 | 61.96 | — | 61.08 |
| | 6.5 | 59.86* | — | 61.13 | 61.13 | 61.96 | — | 60.67 |

*average of 2 measurements
**average of 3 measurements

As can be ascertained from Table 2, the formulations buffers of the invention, i.e. citrate and phosphate buffers had an advantageous effect on Tm compared to other buffers over a range of pH values. The beneficial effect of formulation buffers of the invention was further enhanced by addition of excipients such as NaCl, mannitol and sucrose.

TABLE 3
Melting temperatures (° C.) of 4CXCR104 formulated in different buffers
with different pH and different ionic strengths as determined by TSA.

| buffer (no | | | | | | 66.7 |
|---|---|---|---|---|---|---|
| excipients) | pH | 13.3 mM | 26.7 mM | 40.0 mM | 53.3 mM | mM |
| Citrate | 5.5 | 61.94 | 61.94 | 61.94 | 62.77 | 62.77 |
| | 6.0 | 61.94 | 61.94 | 62.36 | 62.36 | 63.19 |
| | 6.5 | 61.94 | 61.94 | 61.94 | 61.94 | 62.77 |
| phosphate | 6.0 | 61.53 | 61.53 | 61.94 | 62.36 | 62.36 |
| | 6.5 | 61.11 | 61.53 | 61.94 | 61.94 | 61.94 |
| | 7.0 | 61.53 | 61.94 | 61.94 | 62.36 | 62.77 |
| succinate | 5.5 | 60.69 | 61.11 | 61.53 | 61.53 | 61.94 |
| | 6.0 | 61.11 | 61.53 | 61.94 | 61.94 | 61.94 |
| | 6.5 | 61.11 | 61.53 | 61.53 | 61.94 | 61.94 |

Table 3 shows the influence of buffer strength for the buffers of the invention in comparison to one exemplary buffer, succinate. It is shown that higher buffer strengths have a beneficial effect on Tm over a range of pH values. The highest thermal stability was observed in citrate buffer, ph 6.0, between 40 and 66.7 mM buffer concentration.

Overall, 4CXCR104 demonstrated the highest thermal stability in phosphate (pH 6.5-7.0) and citrate buffer (pH 6.0-6.5), while the lowest melting temperatures were recorded in acetate (pH 5.5) and histidine buffer (pH 6.0-6.5) (Table 2). No clear correlation between pH and Tm values was observed across different buffers. In contrast, increasing the buffer strength had a positive effect on thermal stability, as was demonstrated for phosphate, citrate and succinate buffer (Table 3). Also, mannitol, sucrose and high concentrations of NaCl had a stabilizing effect in all buffers.

These data support the advantageous effects in view of clinical applications of 4CXCR104 of the preferred formulation buffers of the invention, i.e. both 50 mM phosphate (pH 7.0) and 50 mM citrate (pH 6.0). In terms of excipients NaCl (widely used buffer component with no negative effect on Tm), mannitol and sucrose (optimal Tm values) are acceptable.

### Tween-80

The advantageous effects of the non-ionic surfactant Tween-80 on physical stability of 4CXCR104 are demonstrated in several stirring experiments performed in 50 mM phosphate (pH 7.0) and 50 mM citrate buffer (pH 6.0). The effect of different concentrations of Tween-80 (no Tween-80 vs. 0.01% vs. 0.05% w/w) on the physical stability of 4CXCR104 was evaluated at 1 mg/mL (by monitoring 500 nm scatter signal at 90° in a spectrofluorometer) and at 10 mg/mL (by monitoring content, turbidity and potential oligomerization). The results are shown in the following table, which depicts the slope of scatter intensity of stirred 4CXCR104 samples and effect of Tween-80. the lower the slope, the lower the increase in the 500 nm scatter signal, i.e. the lower the formation of aggregates.

| | | slope |
|---|---|---|
| | | (absorbance |
| sample no. | buffer composition | units/s) |
| 1 | 50 mM phosphate pH 7.0 | 0.0046000 |
| 2 | 50 mM phosphate pH 7.0 + 0.01% Tween-80 | −0.0000003 |
| 3 | 50 mM phosphate pH 7.0 + 0.02% Tween-80 | 0.0002000 |
| 4 | 50 mM citrate pH 6.0 | 0.0051000 |
| 5 | 50 mM citrate pH 6.0 + 0.01% Tween-80 | 0.0008000 |
| 6 | 50 mM citrate pH 6.0 + 0.02% Tween-80 | 0.0008000 |

At 1 mg/mL, Tween-80 completely prevented an increase in scatter signal in both buffers. No significant differences were observed between samples containing 0.01% or 0.05% Tween-80.

At 10 mg/mL, citrate and phosphate formulations of 4CXCR104 (with or without Tween-80) became turbid after more than 1 hour vigorous stirring (performed in small glass vials on 200 μL volume using maximal stirring power at +4° C.). However, after 1/10 dilution no turbidity could be detected by UV spectrophotometry (A340). Furthermore, all samples became clear again after overnight storage at +4° C. and no oligomers were detected by SE-HPLC suggesting the formation of reversible aggregates. No clear effect of Tween-80 could be demonstrated at 10 mg/mL.

However, protein recovery in citrate buffer (±100%) was significantly better than in phosphate buffer (±83%). This beneficiary effect of citrate was confirmed during further stability testing.

Based on the results described here, in one preferred embodiment the formulation of 4CXCR104 may include 0.01% or 0.05%, preferably 0.01% Tween-80 (w/w).

### 4CXCR104 is Stable in Formulation Buffers of the Invention

Further stability testing of 4CXCR104 shows six formulation buffers of the invention and one control (Table 4). Prior to formulation, the osmolality of several phosphate and citrate buffer solutions with varying strength and excipient concentrations was determined. Based on these results, buffer/excipient concentrations were chosen to obtain isotonic solutions (e.g. 290±20 mOsm/kg). A low concentration of Tween-80 (0.01% w/w) was added to all buffers to protect against mechanical stress (see Tween-80 section above). A formulation in D-PBS was included for comparison.

TABLE 4
Overview of different formulation buffers used in stability
testing of 4CXCR104 (10 mg/mL).

| | | | theoretical | experimental |
|---|---|---|---|---|
| Buffer | buffer | Buffer | osmolality | osmolality |
| no.* | composition** | pH | (mOsm/kg)*** | (mOsm/kg) |
| 1 | 50 mM phosphate + | 7.0 | 288 | 303 |
| | 100 mM NaCl | | | |
| 2 | 50 mM phosphate + | 7.0 | 280 | 285 |
| | 3.0% mannitol | | | |
| 3 | 50 mM phosphate + | 7.0 | 290 | 310 |
| | 6.0% sucrose | | | |
| 4 | 50 mM citrate + | 6.0 | 286 | 288 |
| | 75 mM NaCl | | | |
| 5 | 50 mM citrate + | 6.0 | 285 | 264 |
| | 2.0% mannitol | | | |
| 6 | 50 mM citrate + | 6.0 | 291 | 273 |
| | 4.0% sucrose | | | |
| 7 | D-PBS | 7.4 | — | 302 |

*numbering will be used in following sections (figures and tables)
**all formulations contain 0.01% Tween-80 (w/w)
***calculated based on contribution of buffer, NaCl and excipients

Freeze-Thaw Stability

The effect of repetitive FT cycles on the recovery and physicochemical stability of 4CXCR104 was evaluated. Aliquots of the different formulations (0.2 mL/eppendorf tube) were subjected to up to 10 FT cycles at −70° C. or −20° C. One cycle included freezing for ±30 min followed by thawing in a water bath at +25° C. Treated samples were compared with reference material, which was stored at −70° C. (i.e. 1 FT cycle). Appearance testing showed that the samples looked clear after FT treatment. RP-HPLC and SE-HPLC integration data are summarized in Table 5 and Table 6.

TABLE 5
Integration data from RP-HPLC and SE-HPLC analysis o f4CXCR104 subjected to 1 and
10 FT cycles at −70° C. All test values are comparable to reference.

| | RP-HPLC | | | | | | | SE-HPLC | | |
|---|---|---|---|---|---|---|---|---|---|---|
| FT @ | 1XFT (ref) | | | 10XFT | | | | 1XFT | 10XFT | |
| −70° C. | % | % | % | % | % | % | % | (ref) | | % |
| buffers | pre | main | post | pre | main | post | recovery | % main | % main | recovery |
| 1 | 2.5 | 94.5 | 3.0 | 2.4 | 94.6 | 3.0 | 100.0 | 100.0 | 100.0 | 100.0 |
| 2 | 2.6 | 94.3 | 3.1 | 2.3 | 94.7 | 3.0 | 100.0 | 100.0 | 100.0 | 100.0 |
| 3 | 1.8 | 95.3 | 2.9 | 2.6 | 94.4 | 3.0 | 100.0 | 100.0 | 100.0 | 99.5 |
| 4 | 2.2 | 94.6 | 3.2 | 2.8 | 94.4 | 2.8 | 100.0 | 100.0 | 100.0 | 100.0 |
| 5 | 2.2 | 94.7 | 3.1 | 2.4 | 94.7 | 2.9 | 100.0 | 100.0 | 100.0 | 95.4 |
| 6 | 2.4 | 94.4 | 3.2 | 2.1 | 95.2 | 2.7 | 100.0 | 100.0 | 100.0 | 100.0 |
| 7 | 1.9 | 95.5 | 2.6 | 2.2 | 95.4 | 2.4 | 100.0 | 100.0 | 100.0 | 100.0 |

TABLE 6
Integration data from RP-HPLC and SE-HPLC analysis of 4CXCR104 subjected to 1 and
10 consecutive FT cycles at −20° C. All values were comparable to reference.

| | RP-HPLC | | | | | | | SE-HPLC | | |
|---|---|---|---|---|---|---|---|---|---|---|
| FT @ | 1XFT (ref) | | | 10XFT | | | | 1XFT | 10XFT | |
| −20° C. | % | % | % | % | % | % | % | (ref) | % | % |
| buffers | pre | main | post | pre | main | post | recovery | % main | main | recovery |
| 1 | 2.2 | 94.8 | 3.0 | 2.2 | 95.0 | 2.8 | 100.0 | 100.0 | 100.0 | 100.0 |
| 2 | 2.7 | 94.4 | 2.9 | 2.6 | 94.6 | 2.8 | 99.9 | 100.0 | 100.0 | 100.0 |
| 3 | 2.1 | 95.0 | 2.9 | 2.2 | 95.0 | 2.8 | 100.0 | 100.0 | 100.0 | 100.0 |
| 4 | 2.4 | 94.6 | 3.0 | 2.4 | 94.6 | 3.0 | 97.2 | 100.0 | 100.0 | 100.0 |
| 5 | 2.4 | 94.7 | 2.9 | 2.1 | 94.8 | 3.1 | 100.0 | 100.0 | 100.0 | 99.8 |
| 6 | 2.5 | 94.4 | 3.1 | 2.2 | 95.0 | 2.8 | 99.5 | 100.0 | 100.0 | 100.0 |
| 7 | 2.4 | 94.9 | 2.7 | 2.3 | 94.9 | 2.8 | 100.0 | 100.0 | 100.0 | 100.0 |

Tables 5 and 6 provide evidence that 4CXCR104 demonstrated excellent freeze/thaw stability in all buffers of the invention. FT cycles did not affect the physicochemical properties of the molecule: the RP-HPLC and SE-HPLC profiles of the reference samples (1 FT cycle) and the samples subjected to 10 FT cycles were identical and recoveries were comparable for the different buffers (95-100%).

Stirring Stability

The different 4CXCR104 formulations (see Table 4—all containing 0.01% w:w Tween-80) were subjected to vigorous stirring in small glass vials at +4° C.; concentration=10 mg/mL; volume=200 μL). After 2 hours, all samples became slightly opalescent and RP-HPLC and SE-HPLC analysis was performed. An overview of the integration values is given in Table 7.

Although stirring of 4CXCR104 resulted in protein loss (potentially due to precipitation), no effect on its RP-HPLC or SE-HPLC profile could be detected in any of the formulations (no degradation or oligomerization was observed). Overall, the best recovery was obtained in D-PBS (90.3-94.9%) followed by citrate buffer (76.1-98.3%) and phosphate buffer (57.7-70.6%). Some variation in recovery was seen between the different excipients, although no clear correlation could be made, e.g. in citrate buffer the presence of mannitol resulted in the highest recovery (89.6-98.3%) while in phosphate buffer this excipient gave the lowest recovery (57.7-63.1%).

TABLE 7
Integration data from RP-HPLC and SE-HPLC analysis of 4CXCR104 before and
after 2 hours stirring at +4° C.

| stirring | RP-HPLC | | | SE-HPLC | | |
|---|---|---|---|---|---|---|
| @ +4° | start | stirred | | start | stirred | |
| C. | area | area | % | area | area | % |
| buffers | (mAU * min) | (mAU * min) | recovery | (mAU * min) | (mAU * min) | recovery |
| 1 | 1808.5 | 1224.2 | 67.7 | 51.2 | 36.1 | 70.5 |
| 2 | 1988.2 | 1254.6 | 63.1 | 62.4 | 36.0 | 57.7 |
| 3 | 1717.1 | 1211.5 | 70.6 | 47.8 | 35.5 | 74.3 |
| 4 | 1524.7 | 1210.0 | 79.4 | 44.0 | 35.0 | 79.5 |
| 5 | 1395.4 | 1250.1 | 89.6 | 40.7 | 40.0 | 98.3 |
| 6 | 1572.2 | 1196.4 | 76.1 | 44.6 | 35.3 | 79.1 |
| 7 | 1373.0 | 1302.5 | 94.9 | 39.0 | 35.2 | 90.3 |

AU = Absorption unit

Storage Stability

4CXCR104 was formulated at 10 mg/mL in 6 formulation buffers of the invention (Table 4) and stored at −70° C., +5° C., +25° C. and +40° C. Analysis was performed using the analytical methods described in Table 1 on the following samples:
After 1 month on samples stored at −70° C. and +40° C.
After 7 weeks on samples stored at −70° C., +5° C., +25° C. and +40° C.

Integration results from RP-HPLC and SE-HPLC analysis are summarized in the following table.

TABLE 8
Integration data from RP-HPLC and SE-HPLC analysis of 4CXCR104 formulated in buffer 1-6 and stored
for 7 weeks at −70° C., +5° C., +25° C. and +40° C. An overview of the buffer compositions is given in Table 4.
Values deviating from the control are shown in shades of grey. Protein recovery calculated based on results
from 4CXCR104 sample stored at −70° C., indicated by [100].

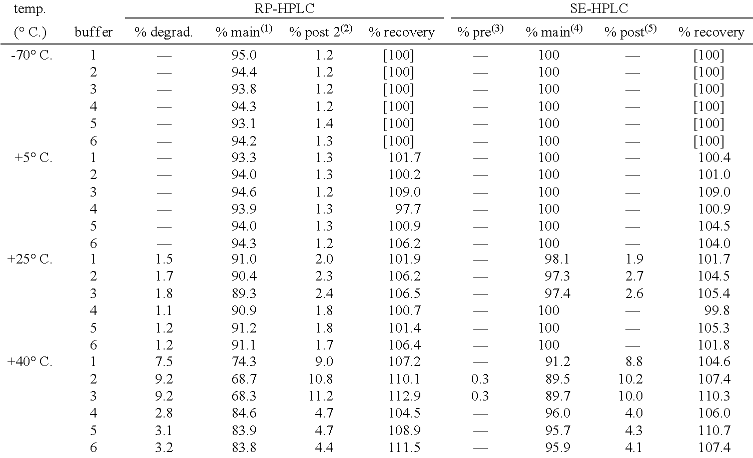

⁽¹⁾unmodified, intact material;
⁽²⁾pyroglutamate variant;
⁽³⁾soluble oligomeric material;
⁽⁴⁾monomeric material;
⁽⁵⁾degradation products

Storage of 4CXCR104 at −70° C. for 7 weeks did not affect its physicochemical characteristics for any of the 6 buffers tested here. Storage did not have a significant effect on RP-HPLC, SE-HPLC or cIEF profiles.

Storage of 4CXCR104 at +5° C. for 7 weeks did not affect its physicochemical characteristics for any of the 6 buffers tested here. Storage did not have a significant effect on its RP-HPLC or SE-HPLC profile.

Storage of 4CXCR104 at +25° C. for 7 weeks did not appear to have an effect on protein recovery, although storage resulted in a slight increase in the amount of pyroglutamate variant and minor protein degradation. This storage effect was more pronounced in phosphate buffers (buffer 1-3) than in citrate buffers (buffer 4-6). SE-HPLC analysis did not detect any oligomers. No significant difference between excipients could be observed.

FIG. 4 shows the absorbance values ((A), A280) and aggregation indices (B) [(100×A340)/(A280-A340)] of 4CXCR104 stored at −70° C. or +40° C. for up to 7 weeks in the 6 potential formulation buffers. Storage at +40° C. caused a minor increase in sample concentration (possibly evaporation). No turbidity was detected for any of the tested buffers.

### RP-HPLC

RP-HPLC results of 4CXCR104 stored at +40° C. for up to 7 weeks in the 6 potential formulation buffers:
Storage under stressed conditions (+40° C.) did not appear to have a significant effect on protein recovery. The obtained surface areas were comparable with that of the reference.
Storage under stressed conditions (+40° C.) caused a gradual increase in degradation products and in the amount of pyroglutamate variant as well as other variants (increasing surface area of pre and post peaks.
Corresponding with the results obtained at +25° C., the effect of storage on the RP-HPLC profile of 4CXCR104 was more pronounced in phosphate buffers (buffer 1-3) than in citrate buffers (buffer 4-6). FIGS. 5 (A) and (B) shows a graphic representation of the kinetics of pyroglutamate formation and degradation in the 6 buffers.
Again, no significant difference between excipients could be observed.

### SE-HPLC

4CXCR104 stored at +40° C. for up to 7 weeks in the 6 formulation buffers was analyzed by SE-HPLC:
Storage under stressed conditions (+40° C.) did not appear to have a significant effect on protein recovery: the surface areas for the stressed samples were comparable with that of reference (see the preceding table).
Storage under stressed conditions (+40° C.) caused a gradual increase in degradation products (increasing surface area of post peaks) which was more pronounced in phosphate buffers (buffer 1-3) than in citrate buffers (buffer 4-6) (confirming the results obtained at +25° C., and the results obtained by RP-HPLC).
A small population of oligomers was detected in buffer 2 and buffer 3 (sucrose) indicating that a formulation of phosphate with mannitol or phosphate with sucrose is more prone to oligomerization.
   cIEF

4CXCR104 stored at +40° C. for up to 1 month in the 6 formulation buffers was analyzed by cIEF electropherograms:
Stressed storage at +40° C. did not appear to have a significant effect on protein recovery (resulting surface areas comparable with that of reference).
Stressed storage at +40° C. caused an increase in pre and post peaks which was more pronounced in phosphate buffers (buffer 1-3) than in citrate buffers (buffer 4-6).
The highest purity was observed in the citrate buffers, confirming SE-HPLC and RP-HPLC data.

### Compatibility

4CXCR104 was formulated at 10 mg/mL in 50 mM citrate pH 6.0 followed by 1/10 and 1/20 dilution in 50 mM citrate (pH 6.0), or 0.9% NaCl and 5 hour incubation at RT. The effect of dilution on appearance, content, pH and chemical stability was negligible (Table 9).

TABLE 9
Compatibility of 4CXCR104 with citrate buffer, or 0.9% NaCl.
Protein recovery calculated based on results from 4CXCR104
diluted in citrate buffer, indicated by [100].

| | | target | | | content | RP-HPLC |
|---|---|---|---|---|---|---|
| | Dilution | conc. | | | (% | (% |
| Diluent | factor | (mg/mL) | visual | pH | recovery) | recovery) |
| 50 mM | 10 | 1.00 | clear | 5.97 | [100] | [100] |
| citrate* | 20 | 0.50 | clear | 5.97 | [100] | [100] |
| 0.9% | 10 | 1.00 | clear | 5.90 | 100 | 100 |
| NaCl** | 20 | 0.50 | clear | 5.89 | 100 | 100 |

*pH 5.98;
**pH 5.54;

A follow-up experiment evaluated the use of formulation buffer vs. 0.9% NaCl as diluent. 4CXCR104 formulated at 10 mg/mL in 50 mM citrate+75 mM NaCl+0.01% Tween-80 (w/w) was diluted 1/5, 1/20 and 1/200 followed by 24 h storage at +4° C. vs. +25° C. (Table 10). Overall, dilution in formulation buffer or 0.9% NaCl did not have a significant effect on sample appearance, pH or its physicochemical characteristics (based on visual inspection, content, RP-HPLC and SE-HPLC). However, dilution in 0.9% NaCl to a final concentration of 0.05 mg/mL resulted in significant protein loss (±80% recovery based on RP-HPLC and SE-HPLC). This drop can most likely be attributed to stickiness of the protein to the container at low concentrations.

TABLE 10
Compatibility study of 4CXCR104 with citrate buffer vs. 0.9% NaCl. Values deviating from reference are
annotated in boldface. Protein recovery calculated based on results from undiluted 4CXCR104 sample,
indicated by [100].

| | | dilution | target conc. | | | content | RP-HPLC | SE-HPLC |
|---|---|---|---|---|---|---|---|---|
| storage | Diluent | factor | (mg/mL) | visual | pH | (% recovery) | (% recovery) | (% recovery) |
| start | (undiluted) | — | 10.00 | clear | 6.00 | [100] | [100] | N.T. |
| | formulation | 5 | 2.00 | clear | 5.88 | 97.9 | 103.2 | N.T. |
| | buffer* | 20 | 0.50 | clear | 5.85 | 98.4 | 102.4 | N.T. |
| | | 200 | 0.05 | clear | 5.83 | N.T. | 99.9 | N.T. |
| | 0.9% NaCl** | 5 | 2.00 | clear | 6.02 | 94.4 | 97.1 | N.T. |
| | | 20 | 0.50 | clear | 6.02 | 94.8 | 99.8 | N.T. |
| | | 200 | 0.05 | clear | 5.92 | N.T. | 81.5 | N.T. |
| 24 h @ | (undiluted) | — | 10.00 | clear | N.T. | [100] | [100] | [100] |
| +4° C. | formulation | 5 | 2.00 | clear | N.T. | 96.8 | 97.9 | 97.1 |
| | buffer* | 20 | 0.50 | clear | N.T. | 97.2 | 98.5 | 98.1 |
| | | 200 | 0.05 | clear | N.T. | N.T. | 97.0 | 98.8 |
| | 0.9% NaCl** | 5 | 2.00 | clear | N.T. | 94.4 | 94.6 | 94.7 |
| | | 20 | 0.50 | clear | N.T. | 94.8 | 96.7 | 96.5 |
| | | 200 | 0.05 | clear | N.T. | N.T. | 81.5 | 79.7 |
| 24 h @ | (undiluted) | — | 10.00 | clear | N.T. | [100] | [100] | [100] |
| +25° C. | formulation | 5 | 2.00 | clear | N.T. | 97.9 | 97.9 | 96.8 |
| | buffer* | 20 | 0.50 | clear | N.T. | 98.8 | 98.9 | 96.4 |
| | | 200 | 0.05 | clear | N.T. | N.T. | 97.0 | 98.7 |
| | 0.9% NaCl** | 5 | 2.00 | clear | N.T. | 96.5 | 95.5 | 93.8 |
| | | 20 | 0.50 | clear | N.T. | 98.8 | 97.2 | 96.4 |
| | | 200 | 0.05 | clear | N.T. | N.T. | 81.4 | 78.5 |

*pH 5.84;
**pH 5.19
N.T. = not tested

### Effects of Most Preferable Formulation Buffer of the Invention

From the above data it is clear that a better physicochemical stability is obtained in citrate buffer than in phosphate buffer for the 4CXCR104 Nanobody® as determined e.g. by RP-HPLC, SE-HPLC and cIEF as detailed above. With regard to freeze-thaw and storage stability, no significant differences between citrate formulations containing NaCl, mannitol or sucrose could be observed. In addition, 4CXCR104 was found to be compatible with 0.9% NaCl to a dilution of 0.5 mg/mL. Based on these results, NaCl can be included in one preferred embodiment as final excipient for obtaining an isotonic formulation.

### Most Preferred Embodiment

In conclusion of all formulation and stability studies performed on the 4CXCR104 Nanobody®, a particularly preferable formulation buffer was defined as:

| | |
|---|---|
| 50 mM citrate, pH 6.0 | 1.325 g/L citric acid monohydrate |
| | 12.850 g/L tri-sodium citrate dehydrate |
| 75 mM NaCl | 4.383 g/L |
| 0.01% Tween-80* | w:w |

*from a 10% stock solution prepared in formulation buffer without Tween-80

### Experimental Series 2

The studies described in this experimental series were aimed at obtaining long-term stability and in use stability data of the liquid formulation of the 4CXCR104 Nanobody®. Experiments described herein below evaluated the effect of:
up to 10 repetitive freeze-thaw (FT) cycles from −70° C. to RT or from −20° C. to RT at 10 mg/mL vs. 0.5 mg/mL (dilution performed in formulation buffer);
storage at 10 mg/mL at −20° C., +5° C., +25° C. and +40° C. for 24 months and even more than 24 months, such as e.g. 36 months or more (data up to 3 months, 6 months and 9 months). 4CXCR104 remains stable at −70° C., −20° C., +5° C. and +25° C. for up to 9 months. At +40° C.±10% degradation and ±11% pyroglutamate formation are observed (remaining potency≈70%) after 6 months and ±13% degradation and ±15% pyroglutamate formation are observed (remaining potency≈61%) after 9 months;
dilution in formulation buffer in glass vials followed by limited storage at +4° C. to generate supportive in use stability data for toxicology studies.

This experimental series pertains to stability data obtained for 4CXCR104 formulated at 10 mg/mL in 50 mM citrate+75 mM NaCl+0.01% Tween-80 (w/w) at pH 6.0. The experiment reports (1) the effect of freeze/thaw (FT) cycles at −20° C. and −70° C. (using diluted and undiluted material), (2) the effect of storage at −70° C., −20° C., +5° C., +25° C. and +40° C., and (3) the effect of dilution in formulation buffer followed by short term storage in glass vials. All experiments were performed to assess the physicochemical stability and potency of the 4CXCR104 Nanobody®.

The following analyses were performed to assess the physicochemical stability and potency:
appearance
content (A280/A340)
reversed-phase chromatography (RP-HPLC)
size exclusion chromatography (SE-HPLC)
capillary isoelectric focusing (cIEF)
potency assay for CXCR4 inhibition as described above

The data generated show that the overall stability of the 4CXCR104 Nanobody® is not affected by 10 freeze-thaw (FT) cycles at −20° C. and −70° C. (at 10 mg/mL or at 0.5 mg/mL). 4CXCR104 remains stable for at least 3 months and even 6 months at −70° C., −20° C., +5° C. and +25° C. While storage at +40° C. resulted in noticeable changes in chemical and physical stability, the Nanobody® kept its product characteristics for purity and homogeneity for all analytical tests (including potency) with the exception of cIEF. Diluting 4CXCR104 in formulation buffer in glass vials followed by short term storage did not have a significant effect on the content of the batch.

### Storage Conditions

The samples in the freeze/thaw stability and storage stability studies were kept in freezers (−70° C.±10° C. and −20° C.±5° C.), a cold room (5° C.±3° C.) and stability chambers (25° C.±2° C. and 40° C.±2° C.) that are not specifically intended for stability studies. At the time of analysis, the samples were transferred to a cold room (5° C.±3° C.) and analyzed as soon as possible (within 1 day, unless described otherwise).

### Product Characteristics for Purity and Homogeneity

Table 11 gives an overview of the product characteristics for purity and homogeneity for the 4CXCR104 Nanobody® as referred to herein. Samples that fulfill the below criteria were considered to be within the product characteristics.

TABLE 11
Product characteristics for purity and homogeneity of
4CXCR104.

| Analysis | Characteristic |
|---|---|
| RP-HPLC | ≧80.0% main peak, e.g. ≧90.0% main peak, preferably |
| | ≧95.0% main peak |
| SE-HPLC | ≧90.0% main peak;; e.g. ≧95.0% main peak |
| cIEF | ≧80.0% main peak; ≧90.0% main peak |
| potency | 50-150% relative to reference standard; e.g. 60-120% |
| | or 70-100%, preferably 80-100% |

### Results

Freeze-Thaw Stability

The effect of repetitive FT cycles on the recovery and physicochemical stability of 4CXCR104 was evaluated. Aliquots of 4CXCR104 formulated at 10 mg/mL and 0.5 mg/mL (1.0 mL/Eppendorf tube) were subjected to up to 10 FT cycles at −70° C. or −20° C. One cycle included freezing for ±30 min followed by thawing in a water bath at +25° C. until all ice crystals were dissolved. Treated samples (5 or 10 FT cycles) were compared with reference material—which was stored at −70° C., i.e. 1 FT cycle—by means of RP-HPLC, SE-HPLC (Table 12) and potency assay (Table 13).

Content was evaluated based on the results obtained from RP-HPLC and SE-HPLC analysis. Freezing and thawing of 4CXCR104 at 10 mg/mL or 0.5 mg/mL did not have a significant effect on content. All samples were visually clear after the treatment.

TABLE 12
Overview of RP-HPLC and SE-HPLC integration data (recovery) and potency data
of 4CXCR104 subjected to 1, 5 and 10 FT cycles at −70° C. and −20° C.

| | | | RP-HPLC | | SE-HPLC | | potency |
|---|---|---|---|---|---|---|---|
| | | | total | | total | | Rel. |
| Temp. | conc. | #FT | area | recovery | area | recovery | act. |
| (° C.) | (mg/mL) | cycles | (mAU * s) | (%) | (mAU * s) | (%) | (%) |
| −70° C. | 10 | 1 | 3552.1 | [100] | 1471.4 | [100] | N.T. |
| <-> | | 5 | 3726.1 | 105 | 1510.3 | 103 | N.T. |
| +25° C. | | 10* | 2487.5 | 70.0 | 1008.0 | 68.5 | 89.4 |
| | 0.5 | 1 | 3528.8 | [100] | 1452.4 | [100] | N.T. |
| | | 5 | 3773.0 | 107 | 1547.5 | 107 | N.T. |
| | | 10 | 3806.8 | 108 | 1553.1 | 107 | 115** |
| −20° C. | 10 | 1 | 3712.5 | [100] | 1526.2 | [100] | N.T. |
| <-> | | 5 | 3861.6 | 104 | 1347.0 | 88.3 | N.T. |
| +25° C. | | 10 | 3512.6 | 94.6 | 1389.8 | 91.1 | 98.7 |
| | 0.5 | 1 | 3224.8 | [100] | 1351.9 | [100] | N.T. |
| | | 5 | 4035.9 | 125 | 1546.6 | 114.4 | N.T. |
| | | 10 | 4123.5 | 128 | 1498.6 | 110.9 | 110** |

*deviating surface area observed due to dilution error prior to HPLC analysis
**samples were stored at +4° C. for 1 week prior to potency analysis
AU = Absorption unit

Potency analysis was performed on 4CXCR104 samples after 10 FT cycles at −70° C. or −20° C. The results of the assay are summarized below.

TABLE 13
Potency data of 4CXCR104 at 10 mg/mL and 0.5 mg/mL subjected
to 10 FT cycles at −70° C. and −20° C. Reference = 4CXCR104
stored at −70° C.

| | |
|---|---|
| 10 mg/mL | 0.5 mg/mL |

| 10 FT cycles | −70° C. | −20° C. | −70° C. | −20° C. |
|---|---|---|---|---|
| relative potency (%) | 89.4 | 98.7 | 114.8 | 110.3 |
| lower limit CI (%) | 77.2 | 76.7 | 98.0 | 88.4 |
| upper limit CI (%) | 110.6 | 127.0 | 134.4 | 137.6 |
| relative CI (%) | 42.8 | 50.9 | 31.8 | 44.6 |

CI = confidence interval

### Conclusion

4CXCR104 demonstrated excellent freeze/thaw stability at 10 mg/ml and 0.5 mg/ml. FT cycles did not affect the physicochemical properties of the molecule: the RP-HPLC and SE-HPLC profiles of the reference samples (1 FT cycle) and the samples subjected to 5 or 10 FT cycles were identical and recoveries were comparable. Moreover, 10 FT cycles did not have a significant effect on the biological activity of 4CXCR104.

Storage Stability

4CXCR104 formulated at 10 mg/mL was stored at −70° C. (reference), +5° C., +25° C. and +40° C. The resulting content, RP-HPLC, SE-HPLC and cIEF profiles and potency data up to 3 months are described in the following. 4CXCR104 remained stable at −70° C., −20° C., +5° C. and +25° C. for up to 9 months. At +40° C. after storage for 6 months, ±10% degradation and ±11% pyroglutamate formation are observed (remaining potency≈70%); at 40° C. after storage for 9 months, ±13% degradation and ±15% pyroglutamate formation are observed (remaining potency≈61%).

Content and Appearance: A slight increase in concentration was observed after storage at +40° C. (possibly due to evaporation). All samples stored at −70° C., −20° C., +5° C. or +25° C. were visually clear and no increase in turbidity was detected by UV spectrophotometry (measured at 340 nm). Samples stored at +40° C. were slightly turbid after 6-9 months, with some minor precipitation observed after 9 months.

Potency analysis was performed on several 4CXCR104 samples. The results of the assay are Table 15.

TABLE 14
Integration data from RP-HPLC, SE-HPLC and cIEF analysis of 4CXCR104 storage samples
formulated at 10 mg/mL.

| | | SE-HPLC | | | RP-HPLC | | | | | |
|---|---|---|---|---|---|---|---|---|---|---|
| temp. | # | % | % | % | % | % | % | % | % | % |
| (° C.) | months | pre⁽¹⁾ | main⁽²⁾ | post⁽³⁾ | degrad.⁽³⁾ | pre 1 | pre 2 | main⁽⁴⁾ | post 1 | post 2⁽⁵⁾ |
| start | 0 | 0.2 | 99.8 | 0.0 | 0.0 | 0.6 | 2.4 | 95.6 | 0.0 | 1.2 |
| −70° C. | 1 | 0.0 | 100 | 0.0 | 0.0 | 0.9 | 2.4 | 95.3 | 0.0 | 1.1 |
| | 2 | NT | | | 0.0 | 0.6 | 2.2 | 95.7 | 0.0 | 1.2 |
| | 3 | 0.0 | 100 | 0.0 | 0.0 | 0.6 | 2.1 | 95.7 | 0.0 | 1.2 |
| | 6 | 0.0 | 100 | 0.0 | 0.0 | 0.7 | 2.1 | 95.7 | 0.0 | 1.1 |
| | 9 | 0.0 | 100 | 0.0 | 0.0 | 0.5 | 2.0 | 96.0 | 0.0 | 1.1 |
| −20° C. | 1 | 0.0 | 100 | 0.0 | 0.0 | 0.9 | 2.8 | 95.0 | 0.0 | 1.1 |
| | 2 | NT | | | NT | | | | | |
| | 3 | 0.0 | 100 | 0.0 | 0.0 | 0.6 | 2.2 | 95.7 | 0.0 | 1.2 |
| | 6 | 0.0 | 100 | 0.0 | 0.0 | 0.7 | 1.8 | 96.2 | 0.0 | 1.0 |
| | 9 | 0.0 | 100 | 0.0 | 0.0 | 0.5 | 2.2 | 95.8 | 0.0 | 1.1 |
| +5° C. | 1 | 0.0 | 100 | 0.0 | 0.0 | 1.0 | 2.4 | 95.2 | 0.0 | 1.1 |
| | 2 | NT | | | NT | | | | | |
| | 3 | 0.0 | 100 | 0.0 | 0.0 | 0.5 | 1.9 | 96.0 | 0.0 | 1.2 |
| | 6 | 0.0 | 100 | 0.0 | 0.0 | 0.8 | 1.8 | 95.8 | 0.0 | 1.2 |
| | 9 | 0.0 | 100 | 0.0 | 0.0 | 0.8 | 2.1 | 95.5 | 0.0 | 1.2 |
| +25° C. | 1 | 0.0 | 100 | 0.0 | 0.0 | 1.0 | 2.8 | 94.7 | 0.0 | 1.2 |
| | 2 | NT | | | NT | | | | | |
| | 3 | 0.0 | 99.1 | 0.9 | 1.3 | 0.8 | 1.7 | 93.6 | 0.0 | 2.0 |
| | 6 | 0.0 | 98.7 | 1.3 | 2.5 | 1.1 | 2.1 | 90.3 | 1.2 | 2.3 |
| | 9 | 0.0 | 97.8 | 2.2 | 2.0 | 1.3 | 1.9 | 90.0 | 1.4 | 3.0 |
| +40° C. | 1 | 0.0 | 98.0 | 2.0 | 1.6 | 1.2 | 1.9 | 90.9 | 1.7 | 2.5 |
| | 2 | NT | | | 2.5 | 1.2 | 2.4 | 87.4 | 2.2 | 4.1 |
| | 3 | 0.1 | 94.9 | 5.0 | 4.2 | 1.7 | 2.6 | 80.1 | 3.6 | 7.4 |
| | 6 | 0.1 | 91.0 | 8.9 | 9.8 | 3.2 | 3.7 | 65.6 | 5.7 | 10.7 |
| | 9 | 0.2 | 85.8 | 14.0 | 12.8 | 3.1 | 4.3 | 56.2 | 6.8 | 15.0 |

| | | RP-HPLC | | cIEF | | | | |
|---|---|---|---|---|---|---|---|---|
| temp. | # | % | % | % | % | % | % | % |
| (° C.) | months | post 3 | post 4 | pre 1 | pre 2 | pre 3 | main | post⁽⁵⁾ |
| start | 0 | 0.2 | 0.0 | 0.0 | 0.0 | 1.7 | 98.3 | 0.0 |
| −70° C. | 1 | 0.3 | 0.0 | 0.0 | 0.0 | 3.3 | 96.7 | 0.0 |
| | 2 | 0.3 | 0.0 | NT | | | | |
| | 3 | 0.3 | 0.1 | 0.0 | 0.0 | 3.6 | 96.4 | 0.0 |
| | 6 | 0.2 | 0.2 | 0.0 | 0.0 | 5.0 | 95.0 | 0.0 |
| | 9 | 0.3 | 0.1 | 0.0 | 0.0 | 5.3 | 94.7 | 0.0 |
| −20° C. | 1 | 0.2 | 0.0 | 0.0 | 0.0 | 4.0 | 96.0 | 0.0 |
| | 2 | NT | | NT | | | | |
| | 3 | 0.2 | 0.1 | 0.0 | P0.0 | 4.3 | 95.7 | 0.0 |
| | 6 | 0.1 | 0.2 | 0.0 | 0.0 | 5.2 | 94.8 | 0.0 |
| | 9 | 0.3 | 0.1 | 0.0 | 0.0 | 5.2 | 94.8 | 0.0 |
| +5° C. | 1 | 0.3 | 0.0 | 0.0 | 0.0 | 2.5 | 97.5 | 0.0 |
| | 2 | NT | | NT | | | | |
| | 3 | 0.3 | 0.2 | 0.0 | 0.0 | 4.1 | 95.9 | 0.0 |
| | 6 | 0.2 | 0.2 | 0.0 | 0.0 | 4.3 | 95.7 | 0.0 |
| | 9 | 0.3 | 0.1 | 0.0 | 0.0 | 5.5 | 94.5 | 0.0 |
| +25° C. | 1 | 0.3 | 0.0 | 0.0 | 0.0 | 4.0 | 96.0 | 0.0 |
| | 2 | NT | | NT | | | | |
| | 3 | 0.4 | 0.2 | 0.0 | 0.0 | 5.2 | 94.0 | 0.8 |
| | 6 | 0.4 | 0.1 | 0.0 | 0.0 | 6.5 | 92.3 | 1.2 |
| | 9 | 0.3 | 0.1 | 0.0 | 0.0 | 7.6 | 90.1 | 2.3 |
| +40° C. | 1 | 0.2 | 0.0 | 0.0 | 0.0 | 7.3 | 89.7 | 3.0 |
| | 2 | 0.2 | 0.0 | NT | | | | |
| | 3 | 0.4 | 0.0 | 3.6 | 4.8 | 13.4 | 73.0 | 5.2 |
| | 6 | 1.1 | 0.2 | 6.3 | 8.0 | 17.2 | 62.0 | 6.5 |
| | 9 | 1.7 | 0.1 | 6.1 | 11.1 | 19.8 | 51.7 | 11.3 |

⁽¹⁾soluble oligomeric material;
⁽²⁾monomeric material;
⁽³⁾ degradation products
⁽⁴⁾unmodified, intact material;
⁽⁵⁾pyroglutamate variant;

TABLE 15
Overview of content, HPLC and potency data of 4CXCR104 storage study.

| | | | | | | | | | RP-HPLC | | | | | | | potency | | |
|---|---|---|---|---|---|---|---|---|---|---|---|---|---|---|---|---|---|---|
| | | | content | | | SE-HPLC | | | % | | | | cIEF | | | % | | |
| temp. | # | | mg/ | | ± | % | % | % | de- | % | % | % | % | % | % | rela- | | ± |
| (° C.) | months | visual | mL | | error | pre | main | post | grad. | pre | main | post | pre | main | post | tive | | error |
| start | 0 | clear | 10.5 | | 0.0 | 0.2 | 99.8 | 0.0 | 0.0 | 3.0 | 95.6 | 1.4 | 1.7 | 98.3 | 0.0 | | NA | |
| | 1 | clear | 10.5 | | 0.0 | 0.0 | 100 | 0.0 | 0.0 | 3.3 | 95.3 | 1.4 | 3.3 | 96.7 | 0.0 | | NA | |
| −70° C. | 2 | clear | | NT | | | NT | | 0.0 | 2.8 | 95.7 | 1.5 | | NT | | | NA | |
| | 3 | clear | 10.8 | | 0.4 | 0.0 | 100 | 0.0 | 0.0 | 2.8 | 95.7 | 1.5 | 3.6 | 96.4 | 0.0 | | NA | |
| | 6 | clear | 11.0 | | 0.0 | 0.0 | 100 | 0.0 | 0.0 | 2.8 | 95.7 | 1.5 | 5.0 | 95.0 | 0.0 | | NA | |
| | 9 | clear | 11.0 | | 0.0 | 0.0 | 100 | 0.0 | 0.0 | 2.5 | 96.0 | 1.5 | 5.3 | 94.7 | 0.0 | | NA | |
| −20° C. | 1 | clear | 10.5 | | 0.3 | 0.0 | 100 | 0.0 | 0.0 | 3.7 | 95.0 | 1.3 | 4.0 | 96.0 | 0.0 | | NT | |
| | 2 | clear | | NT | | | NT | | | | NT | | | NT | | | NT | |
| | 3 | clear | 11.0 | | 0.0 | 0.0 | 100 | 0.0 | 0.0 | 2.8 | 95.7 | 1.5 | 4.3 | 95.7 | 0.0 | | NT | |
| | 6 | clear | 11.1 | | 0.2 | 0.0 | 100 | 0.0 | 0.0 | 2.5 | 96.2 | 1.3 | 5.2 | 94.8 | 0.0 | 108.9 | | 18.5 |
| | 9 | clear | 10.9 | | 1.3 | 0.0 | 100 | 0.0 | 0.0 | 2.7 | 95.8 | 1.5 | 5.2 | 94.8 | 0.0 | | NT | |
| +5° C. | 1 | clear | 10.5 | | 0.3 | 0.0 | 100 | 0.0 | 0.0 | 3.4 | 95.2 | 1.4 | 2.5 | 97.5 | 0.0 | | NT | |
| | 2 | clear | | NT | | | NT | | | | NT | | | NT | | | NT | |
| | 3 | clear | 11.0 | | 0.0 | 0.0 | 100 | 0.0 | 0.0 | 2.4 | 96.0 | 1.6 | 4.1 | 95.9 | 0.0 | | NT | |
| | 6 | clear | 11.0 | | 0.2 | 0.0 | 100 | 0.0 | 0.0 | 2.6 | 95.8 | 1.6 | 4.3 | 95.7 | 0.0 | 112.1 | | 20.1 |
| | 9 | clear | 11.0 | | 0.0 | 0.0 | 100 | 0.0 | 0.0 | 2.9 | 95.5 | 1.6 | 5.5 | 94.5 | 0.0 | | NT | |
| +25° C. | 1 | clear | 10.5 | | 0.3 | 0.0 | 100 | 0.0 | 0.0 | 3.8 | 94.7 | 1.5 | 4.0 | 96.0 | 0.0 | | NT | |
| | 2 | clear | | NT | | | NT | | | | NT | | | NT | | | NT | |
| | 3 | clear | 11.0 | | 0.3 | 0.0 | 99.1 | 0.9 | 1.3 | 2.5 | 93.6 | 2.6 | 5.2 | 94.0 | 0.8 | | NT | |
| | 6 | clear | 11.1 | | 0.0 | 0.0 | 98.7 | 1.3 | 2.5 | 3.2 | 90.3 | 4.0 | 6.5 | 92.3 | 1.2 | | NT | |
| | 9 | clear | 11.1 | | 0.0 | 0.0 | 97.8 | 2.2 | 2.0 | 3.2 | 90.0 | 4.8 | 7.6 | 90.1 | 2.3 | 113.6 | | 17.8 |
| +40° C. | 1 | clear | 10.5 | | 0.3 | 0.0 | 98.0 | 2.0 | 1.6 | 3.1 | 90.9 | 4.4 | 7.3 | 89.7 | 3.0 | | NT | |
| | 2 | clear | | NT | | | NT | | 2.5 | 3.6 | 87.4 | 6.5 | | NT | | | NT | |
| | 3 | clear | 11.3 | | 0.3 | 0.1 | 94.9 | 5.0 | 4.2 | 4.3 | 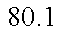 | 11.4 | 21.8 | 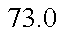 | 5.2 | 84.3 | | 17.2 |
| | 6 | turb | | | 11.9 | 0.2 | 0.1 | 91.8 | 8.9 | 9.8 | 6.9 | 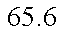 | 17.7 | 31.5 | 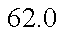 | 6.5 | 70.4 | 10.0 |
| | 9 | turb + p | | | 12.1 | 0.0 | 0.2 | 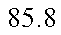 | 14.0 | 12.8 | 7.4 | 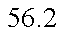 | 23.6 | 37.0 | 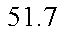 | 11.3 | 61.1 | 9.9 |

Results that did not fulfill the product characteristics with respect to purity and homogeneity are annotated in grey (see Table 11).
NT = not tested;
NA = not applicable;
turb = slightly turbid;
turb + p = slightly turbid and some precipitation

### Conclusion

Storing 4CXCR104 for at least 9 months at −70° C., −20° C. or +5° C. did not significantly affect its physicochemical stability: samples remained clear, content values were stable and RP-HPLC, SE-HPLC and cIEF profiles of the reference material (stored at −70° C.) were comparable with those of the stability samples. No significant effect on total surface area or no additional peaks were observed as a result of storage and 4CXCR104 stayed compliant with product characteristics after 9 months (Table 11).
Storing 4CXCR104 for at least 9 months at +25° C. resulted in minor Nanobody® degradation (seen on SE-HPLC as 2.2% post peak and on RP-HPLC as 2.0% early eluting pre peaks). RP-HPLC analysis also detected a slight increase in pyroglutamate variant with increasing storage time: from 1.2% in the starting material to 3.0% after 9 months. The pyroglutamate variant was also observed on cIEF (2.3% post peak). No significant effect on total surface area was observed as a result of storage and 4CXCR104 stayed compliant with product characteristics after 9 months (Table 11).
Stressing 4CXCR104 for 3 months at +40° C. resulted in significant Nanobody® degradation (seen on SE-HPLC as 14.0% post peak and on RP-HPLC as 12.8% early eluting pre peaks). RP-HPLC analysis also detected an increase in pyroglutamate variant with increasing storage time: from 1.2% in the starting material to 2.5%, 4.1%, 7.4%, 10.7% and 15.0% after 1, 2, 3, 6 and 9 months respectively. The pyroglutamate variant was also observed on cIEF (11.3% post peak after 9 months).
No significant effect on total surface area was observed as a result of storage. Samples stored at −70° C.,-20° C., +5° C. and +25° C. stayed compliant with product characteristics after 9 month storage (Table 11). In addition to the different peaks discussed here, RP-HPLC and cIEF analysis revealed other minor peaks which remain to be identified.

In Use Stability

Previous experiments have demonstrated that dilution of the 4CXCR104 Nanobody® in either formulation buffer or 0.9% NaCl to a final concentration of 0.5 mg/mL followed by up to 24 h storage at +4° C. or +25° C. does not affect its physicochemical stability. Furthermore, the potency data obtained for the FT samples at 0.5 mg/mL demonstrate that an additional storage of 1 week at +4° C. did not have a significant effect on the biological activity of 4CXCR104.

In addition, the effect of storage in glass vials on Nanobody® recovery was evaluated. Several dilutions of 4CXCR104 were prepared in glass 6R vials using formulation buffer. The concentration of the different samples was determined at the start of the experiment and after 24 h storage at +4° C. All samples were clear and storage in glass vials did not appear to have a significant effect on content for any of the dilutions tested (Table 16).

TABLE 16
Overview of content and recovery data of 4CXCR104 during
storage in glass 6R vials. Results were within product
characteristics (see Table 11).

| | Conc. (mg/ml) | | |
|---|---|---|---|
| | T = 0 | T = 24 | % recovery |
| Undiluted | 11.16 | 11.28 | 101.12 |
| Dilution ⅓ | 3.71 | 3.68 | 99.12 |
| Dilution ⅕ | 2.27 | 2.26 | 99.55 |
| Dilution 1/20 | 0.56 | 0.56 | 99.71 |

Further in-use stability testing demonstrated stability throughout different temperature deviations as well as compatibility with different diluents (formulation buffer or saline, tested to 0.3 mg/mL) and passage through catheter or syringe.

### GENERAL CONCLUSION

Subjecting the 4CXCR104 Nanobody® to up to 10 FT cycles at −70° C. or −20° C. at 10 mg/mL or 0.5 mg/mL did not have an effect on its physicochemical properties or its biological activity.
Storage for 9 months at −20° C., +5° C. or +25° C. did not have an effect on its physicochemical properties.
Stressing the Nanobody® at +40° C. for 3 months mainly resulted in protein degradation (4-5% peak area) and formation of the pyroglutamate variant (5-7% peak area), 6 months storage under these conditions resulted in slightly higher values of ±10% peak area degradation and ±11% peak area pyroglutamate formation), 9 months storage under these conditions resulted in higher values of ±13% peak area degradation and ±15% peak area pyroglutamate formation. Samples stored at −70° C., −20° C., +5° C. and +25° C. stayed compliant with product characteristics after 9 month storage (Table 11).
The 4CXCR104 Nanobody® can be diluted in formulation buffer to 0.5 mg/mL and stored in glass 6R vials at +4° C. for up to 24 hours without any significant loss in content.

1. Formulation comprising a polypeptide binding to CXCR4, characterized in that it comprises a citrate or phosphate buffer and has a pH in the range of 5.0 to 7.5.

2. Formulation according to claim 1, wherein the polypeptide comprises at least one immunoglobulin single variable domain binding to CXCR4.

3. Formulation according to claim 1, wherein the polypeptide comprises at least one Nanobody.

4. Formulation according to claim 1, wherein the polypeptide is half-life extended.

5. Formulation according to claim 4, wherein the polypeptide is half-life extended by comprising a polypeptide sequence which binds to serum albumin.

6. Formulation according to claim 5, wherein the polypeptide sequence binding to serum albumin is an immunoglobulin single variable domain or a fragment thereof capable of binding to serum albumin.

7. Formulation according to claim 1, wherein the polypeptide binding to CXCR4 comprises at least one of SEQ ID No. 2 to SEQ ID No. 5, preferably SEQ ID No. 2.

8. Formulation according to claim 1, wherein the buffer has a concentration in the range of 5-100 mM, preferably 5-70 mM, more preferably 5-40 mM, e.g. 10 mM, wherein each value is understood to optionally encompass a range of ±5 mM.

9. Formulation according to claim 1, wherein the formulation has a pH of 5.0, 5.5, 6.0, 6.5, 7.0 or 7.5, preferably 5.5 to 6.5, more preferably 6.0, wherein each value is understood to optionally encompass a range of ±0.2.

10. Formulation according to claim 1, which is suitable for parenteral administration, such as one or more selected from intravenous injection, subcutaneous injection, intramuscular injection or intraperitoneal injection.

11. Formulation according to claim 1, wherein the polypeptide has a concentration in the range of 0.1 to 150 mg/ml, preferably 5-50 mg/ml, such as 5, 10, 15, 20, 25, 30, 35, 40, 45 or 50 mg/ml, preferably 10 mg/ml, wherein each value is understood to optionally encompass a range of ±20% of the specific value.

12. Formulation according to claim 1, further comprising an excipient, which may optionally be one or more selected from the list consisting of NaCl, sucrose or mannitol.

13. Formulation according to claim 1, wherein NaCl has a concentration in the range of 10-500 mM, such as 50, 75, 100, 150, 250 or 500 mM, preferably 25-100 mM, e.g. 75-100 mM; and/or mannitol has a concentration of 1-10%, preferably 2-4%, e.g. 2 or 3% (w/w); and/or sucrose has a concentration of 1-12%, preferably 2-7%, e.g. 4, 5 or 6% (w/w).

14. Formulation according to claim 1, which has an osmolality in the range of 290±60 mOsm/kg.

15. Formulation according to claim 1, wherein the buffer is selected from a) or b)
a) phosphate and preferably has a pH in the range of 6.5 to 7.0, preferably 7.0; or
b) preferably citrate and preferably has a pH between 5.5 and 6.5, more preferably 6.0.

16. Formulation according to claim 1, which further comprises a non-ionic detergent such as Tween-80, preferably in a concentration between 0.005 and 0.1% w/w, more preferably 0.01%.

17. Formulation according to claim 1, wherein the buffer is a citrate buffer at pH 6.0±0.5, e.g. 5.9, 6.0 or 6.1, most specifically 6.0, and the formulation further comprises NaCl, preferably at a concentration of 50-100 mM, e.g. 75 mM, and preferably further comprises a non-ionic detergent such as Tween 80, preferably at a concentration of 0.01%.

18. Formulation according to claim 1, which is in liquid, lyophilized, spray dried or frozen form.

19. Method of preparing a formulation according to claim 1.

20. The method according to claim 19, further comprising a step of confectioning the formulation in a dosage unit form.

21. Method for stabilizing a polypeptide binding to CXCR4, e.g. a polypeptide according to any one of SEQ ID No. 2 to 5, preferably SEQ ID No. 2, for storage, comprising preparing a formulation according to claim 1.

22. (canceled)

23. Method according to claim 21, wherein storage is 1-24 months, such as 1, 3, 6, 9, 12 or 24 months, preferably at least 3 months, e.g. at a temperature between −70° C. and +40° C., such as −70° C., −20° C., +5° C., +25° C. or +40° C., preferably a temperature between −70° C. and +25° C.

24. Pharmaceutical or diagnostic composition comprising a formulation of the polypeptide according to claim 1.

25. (canceled)

26. (canceled)

27. (canceled)

28. (canceled)

29. (canceled)


SEQUENCE LISTING (8 sequences)

SEQ ID NO: 1 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - protein, 270 aa, Artificial Sequence
  Polypeptide
EVQLVESGGGLVQPGGSLRLSCAASGFTFSSYAMSWVRQAPGKGLEWVSGIKSSGDSTRYAGSVKGRFTISRDNAKNTLYLQMNSLRPEDTAVYYCAKSRVSRTGLYTYDNRGQGTLVTVSSGGGGSGGGGSGGGGSGGGGSEVQLVESGGGLVQPGGSLRLSCAASGRTFNNYAMGWFRQAPGKEREFVAAITRSGVRSGVSAIYGDSVKDRFTISRDNAKNTLYLQMNSLRPEDTAVYYCAASAIGSGALRRFEYDYSGQGTLVTVSS

SEQ ID NO: 3 - protein, 270 aa, Artificial Sequence
  Polypeptide
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSGGGGSGGGGSGGGGSGGGGSEVQLVESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 4 - protein, 270 aa, Artificial Sequence
  Polypeptide
EVQLVESGGGLVQTGGSLRLSCAASGFTFSSYAMSWVRQAPGKGLEWVSGIKSSGDSTRYAGSVKGRFTISRDNAKNTLYLQMYSLKPEDTAVYYCAKSRVSRTGLYTYDNRGQGTQVTVSSGGGGSGGGGSGGGGSGGGGSEVQLVESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 5 - protein, 270 aa, Artificial Sequence
  Polypeptide
EVQLVESGGGLVQPGGSLRLSCAASGFTFSSYAMSWVRQAPGKGLEWVSGIKSSGDSTRYAGSVKGRFTISRDNAKNTLYLQMNSLRPEDTAVYYCAKSRVSRTGLYTYDNRGQGTLVTVSSGGGGSGGGGSGGGGSGGGGSEVQLVESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 6 - protein, 122 aa, Artificial Sequence
  Polypeptide
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSS

SEQ ID NO: 7 - protein, 128 aa, Artificial Sequence
  Polypeptide
EVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS

SEQ ID NO: 8 - protein, 270 aa, Artificial Sequence
  Polypeptide
EVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSGGGGSGGGGSGGGGSGGGGSEVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSS